SEQUENCE LISTING (128 sequences)

SEQ ID NO: 1 - protein, 28 aa, synthetic construct
LKKRKVVRLIKFLLKFFLIPKGLVGVFH

SEQ ID NO: 2 - protein, 29 aa, synthetic construct
LKKRKVVRLIKFLLKMFFLIPKGLVGVFH

SEQ ID NO: 3 - protein, 29 aa, synthetic construct
IKKRKVVRLIKFLLKLFFLIPKGLVGVFH

SEQ ID NO: 4 - protein, 29 aa, synthetic construct
VKKRKVVRLIKFLLKLFFLIPKGLVGVFH

SEQ ID NO: 5 - protein, 29 aa, synthetic construct
MKKRKVVRLIKFLLKLFFLIPKGLVGVFH

SEQ ID NO: 6 - protein, 29 aa, synthetic construct
MKKRKVVRLIKYLLKLFFLIPKGLVGVFH

SEQ ID NO: 7 - protein, 29 aa, synthetic construct
IKRRKVVRLIKFLLKLFFLIPKGLVGVFH

SEQ ID NO: 8 - protein, 29 aa, synthetic construct
IKKRKVVRLIKYLLKLFFLIPKGLVGVFH

SEQ ID NO: 9 - protein, 29 aa, synthetic construct
IKKRKVVRLIKKLLKLFFLIPKGLVGVFH

SEQ ID NO: 10 - protein, 29 aa, synthetic construct
IKKRKVLRLIKYLLKLFFLIPKGLVGVFH

SEQ ID NO: 11 - protein, 29 aa, synthetic construct
IKKRKVLRLIKYLVKLFFLIPKGLVGVFH

SEQ ID NO: 12 - protein, 29 aa, synthetic construct
IKKRKIIRLIKFLLKLFFLIPKGLVGVFH

SEQ ID NO: 13 - protein, 29 aa, synthetic construct
IKKRKIVRLIKFLVKLFFLIPKGLVGVFH

SEQ ID NO: 14 - protein, 27 aa, synthetic construct
LKKRKVVRLIKFLLFFLIPKGLVGVFH

SEQ ID NO: 15 - protein, 26 aa, synthetic construct
LKKRKVVRLIKFLFFLIPKGLVGVFH

SEQ ID NO: 16 - protein, 25 aa, synthetic construct
LKKRKVVRLIKFFFLIPKGLVGVFH

SEQ ID NO: 17 - protein, 29 aa, synthetic construct
LKKRKVVRLIKFLLKLLVGVFHFFLIPKG

SEQ ID NO: 18 - protein, 29 aa, synthetic construct
FFLIPKGLKKRKVVRLIKFLLKLLVGVFH

SEQ ID NO: 19 - protein, 29 aa, synthetic construct
VKKRKIVRLIKFLVKLFFLIPKGLVGVFH

SEQ ID NO: 20 - protein, 29 aa, synthetic construct
VKKRKIVRLIKFLVKMFFLIPKGLVGVFH

SEQ ID NO: 21 - protein, 28 aa, synthetic construct
VKKRKIVRLIKFLLKFFLIPKGLVGVFH

SEQ ID NO: 22 - protein, 28 aa, synthetic construct
VKKRKVVRLIKFLLKFFLIPKGLVGVFH

SEQ ID NO: 23 - protein, 25 aa, synthetic construct
VKKRKVVRLIKLFFLIPKGLVGVFH

SEQ ID NO: 24 - protein, 25 aa, synthetic construct
VKKRKVVRLIKYFFLIPKGLVGVFH

SEQ ID NO: 25 - protein, 25 aa, synthetic construct
VKKRKIVRLIKVFFLIPKGLVGVFH

SEQ ID NO: 26 - protein, 28 aa, synthetic construct
VKKRKVVRLIKFVLKFFLIPKGLVGVFH

SEQ ID NO: 27 - protein, 28 aa, synthetic construct
VKKRKVVRLIKFVLKYFLIPKGLVGVFH

SEQ ID NO: 28 - protein, 28 aa, synthetic construct
VKKRKLVRLIKFVLKFFLIPKGLVGVFH

SEQ ID NO: 29 - protein, 28 aa, synthetic construct
VKKRKIVRLIKFVLKFFLIPKGLVGVFH

SEQ ID NO: 30 - protein, 24 aa, synthetic construct
VKKRKIVRLIKFFLIPKGLVGVFH

SEQ ID NO: 31 - protein, 23 aa, synthetic construct
VKKRKIVRLIFFLIPKGLVGVFH

SEQ ID NO: 32 - protein, 28 aa, synthetic construct
VKKRKIVRLIKFLLKFFLIPKGIVGVFH

SEQ ID NO: 33 - protein, 28 aa, synthetic construct
VKKRKIVRLIKFLLKYFLIPKGVVGVFH

SEQ ID NO: 34 - protein, 25 aa, synthetic construct
VKKRKVVRLIKYFFLIPKGVVGVFH

SEQ ID NO: 35 - protein, 25 aa, synthetic construct
VKKRKIVRLIKYFFLIPKGVVGVFH

SEQ ID NO: 36 - protein, 25 aa, synthetic construct
VKKRKIVRLIKYFFLVPKGIVGVFH

SEQ ID NO: 37 - protein, 25 aa, synthetic construct
VKKRKVVRLIKYYFLIPKGIVGVFH

SEQ ID NO: 38 - protein, 24 aa, synthetic construct
VKKRKVIRLIKFFLIPKGLVGVFH

SEQ ID NO: 39 - protein, 24 aa, synthetic construct
VKKRKILRLIKFFLIPKGLVGVFH

SEQ ID NO: 40 - protein, 23 aa, synthetic construct
VKKRKIVRLIKFFLIPKLVGVFH

SEQ ID NO: 41 - protein, 36 aa, synthetic construct
LKKRKVVRLIKFLLKLLKKRKVVFFLIPKGRRRRRR

SEQ ID NO: 42 - protein, 34 aa, synthetic construct
LKKRKVVRLIKFLLKLLKKRKVVFFLIPKGRRRR

SEQ ID NO: 43 - protein, 27 aa, synthetic construct
LKKRKVVRLIKFLLKLFFLIPKGRRRR

SEQ ID NO: 44 - protein, 30 aa, synthetic construct
LKKRKVVRLIKFLLKLLKKFFLIPKGRRRR

SEQ ID NO: 45 - protein, 37 aa, synthetic construct
LKKRKVVRLIKFLLKLLKKFFLIPKGACSSSPSKHCG

SEQ ID NO: 46 - protein, 30 aa, synthetic construct
LKKRKVVRLIKFLLKLFFLIPKGRPARPAR

SEQ ID NO: 47 - protein, 35 aa, synthetic construct
LKKRKVVRLIKFLLKLFFLIPKGTHRPPMWSPVWP

SEQ ID NO: 48 - protein, 38 aa, synthetic construct
LKKRKVVRLIKFLLKLFFLIPKGGYRPVHNIRGHWAPG

SEQ ID NO: 49 - protein, 26 aa, synthetic construct
LKKRKVVRLIKFLLKFFLIPKGRLYE

SEQ ID NO: 50 - protein, 29 aa, synthetic construct
LKKRKVVRLIKFLLKFFLIPKGRPARPAR

SEQ ID NO: 51 - protein, 30 aa, synthetic construct
LKKRKVVRLIKFLLKLFFLIPKGAHLHNRS

SEQ ID NO: 52 - protein, 31 aa, synthetic construct
LKKRKVVRLIKFLLKLFFLIPKGNAHQARST

SEQ ID NO: 53 - protein, 29 aa, synthetic construct
LKKRKVVRLIKFLLKLFFLIPKGLVGVFH

SEQ ID NO: 54 - protein, 30 aa, synthetic construct
LKKRKVVRLIKFLLKLFFLIPKGQHREDGS

SEQ ID NO: 55 - protein, 29 aa, synthetic construct
LKKRKVVRLIKFLLKLFFLIPKGRPLKPW

SEQ ID NO: 56 - protein, 29 aa, synthetic construct
LKKRKVVRLIKFLLKLLVFFLIPKGRLYE

SEQ ID NO: 57 - protein, 30 aa, synthetic construct
LKKRKVVRLIKFLLKLFFLIPKGNLLMAAS

SEQ ID NO: 58 - protein, 28 aa, synthetic construct
LKKRKVVRLIKFLLKLFFLIPKGDKPRR

SEQ ID NO: 59 - protein, 27 aa, synthetic construct
LKKRKVVRLIKFLLKLFFLIPKGRPAR

SEQ ID NO: 60 - protein, 29 aa, synthetic construct
LVGVFHLKKRKVVRLIKFLLKLFFLIPKG

SEQ ID NO: 61 - protein, 28 aa, synthetic construct
LVGVFHLKKRKVVRLIKFLLKFFLIPKG

SEQ ID NO: 62 - protein, 27 aa, synthetic construct
LVGVFHLKKRKVVRLIKFLLFFLIPKG

SEQ ID NO: 63 - protein, 26 aa, synthetic construct
LVGVFHLKKRKVVRLIKFLFFLIPKG

SEQ ID NO: 64 - protein, 25 aa, synthetic construct
LVGVFHLKKRKVVRLIKFFFLIPKG

SEQ ID NO: 65 - protein, 28 aa, synthetic construct
LVGVFHVKKRKIVRLIKFLVKFFLIPKG

SEQ ID NO: 66 - protein, 28 aa, synthetic construct
IVGVFHVKKRKIVRLIKFLVKFFLIPKG

SEQ ID NO: 67 - protein, 25 aa, synthetic construct
LVGVFHVKKRKIVRLIKFFFLIPKG

SEQ ID NO: 68 - protein, 25 aa, synthetic construct
IVGVFHVKKRKIVRLIKFYFLIPKG

SEQ ID NO: 69 - protein, 25 aa, synthetic construct
IVGVFHVKKRKIVRLVKFYFLIPKG

SEQ ID NO: 70 - protein, 25 aa, synthetic construct
LVGVFHVKKRKVVRVIKFFFLIPKG

SEQ ID NO: 71 - protein, 28 aa, synthetic construct
VVGVFHVKKRKIVRLIKFLLKFFLIPKG

SEQ ID NO: 72 - protein, 25 aa, synthetic construct
VVGVFHVKKRKIVRLIKFFFLIPKG

SEQ ID NO: 73 - protein, 6 aa, synthetic construct
RRRRRR

SEQ ID NO: 74 - protein, 9 aa, synthetic construct
RKKRRQRRR

SEQ ID NO: 75 - protein, 35 aa, synthetic construct
KKLFKKILKYLKKLFKKILKYLFFLIPKGRRRRRR

SEQ ID NO: 76 - protein, 41 aa, synthetic construct
LKKRKVVRLIKFLLKLLKKRKVVFFLIPKGACSSSPSKHCG

SEQ ID NO: 77 - protein, 36 aa, synthetic construct
KKLFKKILKYLINLKALAALAKKILRPSRPRIRYKC

SEQ ID NO: 78 - protein, 40 aa, synthetic construct
LKKRKVVRLIKFLLKLLKKRKVVFFLIPKGRRRRRRAVPF

SEQ ID NO: 79 - protein, 44 aa, synthetic construct
LKKRKVVRLIKFLLKLLKKRKVVFFLIPKGMASIWVGHRGAVPF

SEQ ID NO: 80 - protein, 38 aa, synthetic construct
LKKRKVVRLIKFLLKLLKKRKVVFFLIPKGRRRRRLYE

SEQ ID NO: 81 - protein, 34 aa, synthetic construct
LKKRKVVRLIKFLLKLLKKRKVVFFLIPKGRLYE

SEQ ID NO: 82 - protein, 40 aa, synthetic construct
KKLFKKILKYLKKLFKKILKYLRPSRPRIRYKCRPARPAR

SEQ ID NO: 83 - protein, 40 aa, synthetic construct
LKKRKVVRLIKFLLKLLKKRKVVFFLIPKGRRRRPARPAR

SEQ ID NO: 84 - protein, 40 aa, synthetic construct
LKKRKVVRLIKFLLKLLKKRKVVFFLIPKGRRRRRRKRRR

SEQ ID NO: 85 - protein, 45 aa, synthetic construct
LKKRKVVRLIKFLLKLLKKRKFFLIPKGACSSSPSKHCGRRKRRR

SEQ ID NO: 86 - protein, 39 aa, synthetic construct
LKKRKVVRLIKFLLKLLKKRKFFLIPKGACSSSPSKHCG

SEQ ID NO: 87 - protein, 34 aa, synthetic construct
LKKRKVVRLIKNLLKLLKKRKVNFFLIPKGRRRR

SEQ ID NO: 88 - protein, 34 aa, synthetic construct
LKKRKVVRLIKFNLKLLKKRKVNFFLIPKGRRRR

SEQ ID NO: 89 - protein, 34 aa, synthetic construct
LKKRKVVRLIKFLLKLFFLIPKGACSSSPSKHCG

SEQ ID NO: 90 - protein, 29 aa, synthetic construct
KKRKVVRLIKFLLKLLKKRKVVFFLIPKG

SEQ ID NO: 91 - protein, 31 aa, synthetic construct
LKKRKVVRLIKFLLKLLKKRKVVFFLIPKGE

SEQ ID NO: 92 - protein, 32 aa, synthetic construct
LKKRKVVRLIKFLLKLLKKRKVVFFLIPKGLE

SEQ ID NO: 93 - protein, 27 aa, synthetic construct
LKKRKVVRLIKFLLKLFFLIPKGRLYE

SEQ ID NO: 94 - protein, 35 aa, synthetic construct
LKKRKVVRLIKFLLKLFFLIPKGHHIYLGAVNYIY

SEQ ID NO: 95 - protein, 28 aa, synthetic construct
LKKRKVVRLIKFLLKLLKKFFLIPKGLE

SEQ ID NO: 96 - protein, 28 aa, synthetic construct
LKKRKVVRLIKFLLKLRFFLIPKGRLYE

SEQ ID NO: 97 - protein, 18 aa, synthetic construct
KAFDITYVRLKFFLIPKG

SEQ ID NO: 98 - protein, 29 aa, synthetic construct
FFLIPKGLVGVFHLKKRKVVRLIKFLLKL

SEQ ID NO: 99 - protein, 23 aa, synthetic construct
  VARIANT 20: note = I or V
  VARIANT 1..17: note = X can be any amino acid
XXXXXXXXXXXXXXXXXFLXPKG

SEQ ID NO: 100 - protein, 23 aa, synthetic construct
  VARIANT 1..17: note = X can be any amino acid
XXXXXXXXXXXXXXXXXFLIPKG

SEQ ID NO: 101 - protein, 23 aa, synthetic construct
  VARIANT 1: note = L or V
  VARIANT 6: note = I or V
  VARIANT 12..16: note = X can be any amino acid
XKKRKXVRLIKXXXXXFFLIPKG

SEQ ID NO: 102 - protein, 6 aa, synthetic construct
LVGVFH

SEQ ID NO: 103 - protein, 6 aa, synthetic construct
RRRRRR

SEQ ID NO: 104 - protein, 4 aa, synthetic construct
RRRR

SEQ ID NO: 105 - protein, 4 aa, synthetic construct
RYLE

SEQ ID NO: 106 - protein, 7 aa, synthetic construct
RPARPAR

SEQ ID NO: 107 - protein, 14 aa, synthetic construct
MASIWVGHRGAVPF

SEQ ID NO: 108 - protein, 11 aa, synthetic construct
RPSRPRIRYKC

SEQ ID NO: 109 - protein, 6 aa, synthetic construct
RRKRRR

SEQ ID NO: 110 - protein, 5 aa, synthetic construct
ARPCA

SEQ ID NO: 111 - protein, 7 aa, synthetic construct
GVITRIR

SEQ ID NO: 112 - protein, 11 aa, synthetic construct
ACSSSPSKHCG

SEQ ID NO: 113 - protein, 6 aa, synthetic construct
LVGVFH

SEQ ID NO: 114 - protein, 7 aa, synthetic construct
HAIYPRH

SEQ ID NO: 115 - protein, 15 aa, synthetic construct
GYRPVHNIRGHWAPG

SEQ ID NO: 116 - protein, 7 aa, synthetic construct
AHLHNRS

SEQ ID NO: 117 - protein, 8 aa, synthetic construct
NAHQARST

SEQ ID NO: 118 - protein, 7 aa, synthetic construct
QHREDGS

SEQ ID NO: 119 - protein, 7 aa, synthetic construct
NLLMAAS

SEQ ID NO: 120 - protein, 5 aa, synthetic construct
DKPRR

SEQ ID NO: 121 - protein, 5 aa, synthetic construct
RPAPR

SEQ ID NO: 122 - protein, 6 aa, synthetic construct
CNGRCG

SEQ ID NO: 123 - protein, 12 aa, synthetic construct
THRPPMWSPVWP

SEQ ID NO: 124 - protein, 6 aa, synthetic construct
RPLKPW

SEQ ID NO: 125 - protein, 25 aa, synthetic construct
KAFDITYVRLKFFLIPKGRPARPAR

SEQ ID NO: 126 - protein, 27 aa, synthetic construct
VKKRKIVRLIKFFLIPKLVGVFHRLYE

SEQ ID NO: 127 - protein, 34 aa, synthetic construct
KKLKKLKVRLVKFLLKFGPLRYRVVLFIERIKLK

SEQ ID NO: 128 - protein, 26 aa, synthetic construct
KKLKKLKVRLVKFLLKFGPLRYRVVL

(19) United States
(12) Patent Application Publication
(10) Pub. No.: US 2024/0299564 A1
(43) Pub. Date: Sep. 12, 2024

(54) PEPTIDE CARRIERS AND DELIVERY TO THE EYE OF A SUBJECT

(71) Applicant: WINCAL BIOPHARM, INC., South San Francisco, CA (US)

(72) Inventors: Venice Chiueh, San Bruno, CA (US); Linda Lee, South San Francisco, CA (US); TaeWeon Lee, Palo Alto, CA (US)

(21) Appl. No.: 18/596,555

(22) Filed: Mar. 5, 2024

Related U.S. Application Data
(60) Provisional application No. 63/450,192, filed on Mar. 6, 2023.

(51) Int. Cl. A61K 47/64; A61K 9/00; A61P 27/02; C07K 14/00

(52) U.S. Cl. CPC A61K 47/64; A61K 9/0048; A61P 27/02; C07K 14/001

(57) ABSTRACT

The disclosure provides a peptide comprising no more than 50 amino acids and comprising the amino acid sequence wherein the peptide comprises the amino acid sequence of Formula (1): X1-X2-X3-X4-X5-X6-X7-X8-X9-X10-X11-X12-X13-X14-X15-X16-X17-F-L-N/V-P-K-G, including peptides comprising the amino acid sequence of any one of SEQ ID NO: 1-72. The disclosure further provides methods of delivering a payload peptide to an eye of a subject, as well as a method of treating an ocular disorder in a subject in need thereof.

### CROSS REFERENCE TO RELATED APPLICATIONS

This application claims the benefit of priority of U.S. Provisional Patent Application No. 63/450,192, filed Mar. 6, 2023, which is hereby incorporated by reference in its entirety.

### FIELD OF THE INVENTION

The present disclosure is generally directed to peptide carriers, compositions comprising such peptide carriers, and methods of treating an ocular disease.

### INCORPORATION BY REFERENCE OF MATERIAL ELECTRONICALLY SUBMITTED

This application is filed with a sequence listing in electronic form which is incorporated herein by reference in its entirety and identified as follows: Filename: 58450_SeqListing.xml; Size: 116,111 bytes; Created: Feb. 27, 2024

### BACKGROUND

Achieving effective drug delivery to the posterior segment with an eye drop is challenging (Rodrigues, G. A., et al, Pharm Res, 2018. 35(12): p. 245; Singh, R., et al, Retina, 2014. 34(9): p. 1787-95; Nomoto, H., et al, Invest Ophthalmol Vis Sci, 2009. 50(10): p. 4807-13; Iwase, T., et al, Invest Ophthalmol Vis Sci, 2013. 54(1): p. 503-11). The most common approach for achieving drug delivery to the posterior segment is intravitreal (IVT) injection. However, IVT injection is relatively invasive and is associated with rare, but severe ocular complications (Jager, R. D., et al, Retina, 2004. 24(5): p. 676-98; Boyer, D. S., et al, Ophthalmology, 2014. 121(10): p. 1904-14). Current treatments for neovascular age-related macular degeneration (AMD) and diabetic macular edema (DME) include the direct injections of EYLEA® (aflibercept, Regeneron), LUCENTIS® (ranibizumab, Genentech), BEOVU® (brolucizumab, Novartis), or MACUGEN® (pegaptanib, Gilead) into the vitreous region in subject in need thereof. Injections are required, e.g., every 4-8 weeks to deliver biologics for treatment of wet age-related macular degeneration (Avery, R. L., et al, Ophthalmology, 2006. 113(3): p. 363-372 e5; Becerra, E. M., et al, Curr Drug Targets, 2011. 12(2): p. 149-72). As many ocular diseases require treatment for decades, the requirement for invasive injections is a significant inconvenience for patients.

### SUMMARY

The disclosure provides a peptide comprising no more than 50 amino acids and comprising the amino acid sequence of Formula (1): X1-X2-X3-X4-X5-X6-X7-X8-X9-X10-X11-X12-X13-X14-X15-X16-X17-F-L-I/V-P-K-G; wherein X1 is V, M, L, I, or K; X2 is K, R, or H; X3 is K, R, or H; X4 is R, K, or H; X5 is K, R, or H; X6 is I, V, or L; X7 is V, L, or I; X8 is R, K, or H; X9 is L, K, V, or R; X10 is I, L, or V; X11 is K, R, or H; X12 is F, Y, K, or L, or is absent; X13 is L, V, or I, or is absent; X14 is V, L, or I, or is absent; X15 is K, L, R, or H, or is absent; X16 is (i) M, L, V, I, R, A, K, G, C, Y, S, N, or D; is an amino acid of (i) in combination with L, K, R, or V, or (iii) is absent; and X17 is F, Y, FL, YL, FLK, YLK, FLKL, or YLKL. Optionally, the peptide comprises the amino acid sequence of Formula (2): X1-X2-X3-X4-X5-X6-X7-X8-X9-X10-X11-X12-X13-X14-X15-X16-X17-F-L-I-P-K-G. For example, in various aspects, the peptide comprises the amino acid sequence of Formula (3): L/V-K-K-R-K-I/V-V-R-L-I-K-X12-X13-X14-X15-X16-F-F-L-I-P-K-G, wherein X12 is F or Y or is absent; X13 is L or is absent; X14 is L or V or is absent; X15 is K or is absent; and X16 is L or M or is absent. Optionally, the peptide further comprises, attached at the N-terminus, L, L-L, or the amino acid sequence X30-V/L-G-V/L-F-H (Formula (4)), wherein X30 is L, I, or V. Also optionally, the peptide further comprises the amino acid sequence of one of SEQ ID NOs: 103-124 [Z1-Z23, provided in Table A below], attached at the C-terminus. The disclosure further provides a construct comprising the peptide conjugated to one or more payload peptides, a composition comprising one or more peptides and one or more payload peptides, and a composition comprising the construct. A method of delivering a payload peptide to an eye of a subject is provided, wherein the method comprises administering the composition of the disclosure directly to the eye of the subject. A method of treating an ocular disorder in a subject in need thereof also is provided, wherein the method comprises administering the composition of the disclosure to the eye of the subject. The disclosure also provides use of the composition disclosed herein in a method of treating an ocular disorder, as well as use of the composition in the preparation of a medicament for treating an ocular disorder.

It should be understood that, while various embodiments in the specification are presented using “comprising” language, under various circumstances, a related embodiment may also be described using “consisting of” or “consisting essentially of” language. The disclosure contemplates embodiments described as “comprising” a feature to include embodiments which “consist of” or “consist essentially of” the feature. The term “a” or “an” refers to one or more. As such, the terms “a” (or “an”), “one or more,” and “at least one” can be used interchangeably herein. The term “or” should be understood to encompass items in the alternative or together, unless context unambiguously requires otherwise.

It should also be understood that when describing a range of values, the disclosure contemplates individual values found within the range. For example, “a molar ratio from about 1:1 to about 1:70,” could be, but is not limited to, a molar ratio of 1:1, 1:10, 1:50, etc., and any value in between such values. In any of the ranges described herein, the endpoints of the range are included in the range. However, the description also contemplates the same ranges in which the lower and/or the higher endpoint is excluded. When the term “about” is used, it means the recited number plus or minus 5%, 10%, or more of that recited number. The actual variation intended is determinable from the context.

Additional features and variations of the invention will be apparent to those skilled in the art from the entirety of this application, including the figures and detailed description, and all such features are intended as aspects of the invention. Likewise, features of the invention described herein can be re-combined into additional embodiments that also are intended as aspects of the invention, irrespective of whether the combination of features is specified as an aspect or embodiment of the invention. The entire document is intended to be related as a unified disclosure, and it should be understood that all combinations of features described herein (even if described in separate sections) are contemplated, even if the combination of features is not found together in the same sentence, or paragraph, or section of this document. Also, only such limitations which are described herein as critical to the invention should be viewed as such; variations of the invention lacking limitations which have not been described herein as critical are intended as aspects of the invention.

### BRIEF DESCRIPTION OF THE FIGURES

FIG. 1 is a graph showing the results of an intraocular penetration screening of IR800-aflibercept biosimilar with peptide carrier eye drop formulations containing peptide carrier (OPC) 712, 715, 721, 722, 723, 724, 725, 726, 728, 729, 730, 731, 733, 735, 736, 737, 741, 742, 743, 744, 745, 746, 747, 748, 749, 750, 751, 752, 753, 754, 755, 759, 760, 761, 762, 763, 764, 765, 766, 775, 776, or 777 in mouse eyes. Vitreous and retina samples were collected 15 minutes after eye drop application of IR800 labeled aflibercept-PC formulation (1:20 ratios) in Buffer 1 (5 ug in 3 uL per eye). Intraocular IR800-aflibercept signal±SEM, N=2-6 eyes.

FIG. 2 is a graph showing the results of an intraocular penetration screening of IR800-aflibercept biosimilar with peptide carrier eye drop formulations containing peptide carrier (OPC) 381, 498, 596, 597, 608, 633, 638, 640, 641, 642, 651, 653, 654, 656, 658, 664, 698, 700, or 703 in mouse eyes. Vitreous and retina samples were collected 15 minutes after eye drop application of IR800 labeled aflibercept-PC formulation (1:20 ratios) in Buffer 1 (5 ug in 3 uL per eye). Intraocular IR800-aflibercept signal±SEM, N=2-65 eyes.

FIG. 3 is a graph showing the results of an intraocular penetration screening of IR800-aflibercept biosimilar with peptide carrier eye drop formulations containing peptide carrier (OPC) 689, 738, 739, 740, 741, 768, 769, 771, 771, 772, or 773 in mouse eyes. Vitreous and retina samples were collected 15 minutes after eye drop application of IR800 labeled aflibercept-PC formulation (1:20 ratios) in Buffer 1 (5 ug in 3 uL per eye). Intraocular IR800-aflibercept signal±SEM, N=2-12 eyes.

FIG. 4 is a graph showing the results of an intraocular penetration screening of IR-800-brolucizumab with peptide carrier eye drop formulations containing peptide carrier (OPC) 1, 498, 499, 502, 514, 564, 571, 572, 578, 579, 587, 591, 595, 596, 597, 603, 604, 607, 608, 654, 658, 667, 698, 700, 715, 730, 737 or 759 in mouse eyes. Vitreous and retina samples were collected 15 minutes after eye drop application of IR800 labeled brolucizumab-PC formulation (1:10 ratios) in Buffer 1 (3 ug in 3 uL per eye). Intraocular IR800-brolucizumab signal±SEM, N=2-8 eyes.

FIG. 5 is a graph showing the results of intraocular penetration screening of IR800-ranibizumab with peptide carrier eye drop formulations containing peptide carrier (OPC) 1, 9, 498, 529, 564, 572, 578, 579, 596, 597, 604, 608, 622, 623, 624, 631, 633, 634, 638, 640, 641, 651, 653, 654, 656, 658, 664, 667, 670, 689, or 698 in mouse eyes. Vitreous and retina samples were collected 15 minutes after eye drop application of IR800 labeled ranibizumab-PC formulation (1:12 ratios) in Buffer 1 (5 ug in 3 uL per eye). Intraocular IR800-ranibizumab signal±SEM, N=2-4 eyes.

FIG. 6 is a graph showing the results of intraocular penetration screening of IR800-bevacizumab with peptide carrier eye drop formulations containing peptide carrier (OPC) 1, 9, 498, 529, 564, 572, 578, 579, 596, 597, 604, 608, 622, 623, 624, 631, 633, 634, 638, 640, 641, 648, 651, 653, 654, 656, 658, 664, 667, 670, 689, 698, 715, 730, or 737 in mouse eyes. Vitreous and retina samples were collected 15 minutes after eye drop application of IR800 labeled bevacizumab-PC formulation (1:20 ratios) in Buffer 2 (10 ug in 3 uL per eye). Intraocular IR800-bevacizumab signal±SEM, N=2-6 eyes.

FIG. 7 is a graph showing the results of intraocular penetration screening of IR800-faricimab with peptide carrier eye drop formulations containing peptide carrier (OPC) 1, 498, 597, 608, 631, 633, 640, 653, 654, 689, 698, 700, 715, 730, 736, 737, 746, 747, 750, 759, 762, 764, 770, 776, or 777 in mouse eyes. Vitreous and retina samples were collected 15 minutes after eye drop application of IR800 labeled faricimab-PC formulation (1:20 ratios) in Buffer 1 (10 ug in 3 uL per eye). Intraocular IR800-faricimab signal±SEM, N=2-6 eyes.

FIG. 8 is a graph showing the results of intraocular penetration screening of IR800-cetuximab with peptide carrier eye drop formulations containing peptide carrier (OPC) 1, 9, 498, 529, 564, 572, 578, 579, 597, 604, 608, 623, 631, 633, 638, 640, 641, 651, 653, 654, 658, 667, 670, 689, 700, 715, 730, 736, or 737 in mouse eyes. Vitreous and retina samples were collected 15 minutes after eye drop application of IR800 labeled bevacizumab-PC formulation (1:20 ratios) in Buffer 1 (10 ug in 3 uL per eye). Intraocular IR800-brolucizumab signal±SEM, N=2 eyes per test.

FIG. 9 is a graph showing the results of intraocular penetration screening of IR800-ramucirumab with peptide carrier eye drop formulations containing peptide carrier (OPC) 1, 498, 597, 608, 631, 633, 640, 653, 654, 689, 698, 700, 715, 730, 736, 737, 746, 747, 750, 759, 762, 764, 770, 776 or 777 in mouse eyes. Vitreous and retina samples were collected 15 minutes after eye drop application of IR800 labeled ramucirumab-PC formulation (1:20 ratios) in Buffer 1 (10 ug in 3 uL per eye). Intraocular IR800-brolucizumab signal±SEM, N=2 eyes per test.

FIG. 10 is a graph showing the results of intraocular penetration screening of IR800-adalimumab with peptide carrier eye drop formulations containing peptide carrier (OPC) 1, 9, 498, 502, 578, 579, 596, 597, 604, 608, 623, 624, 631, 633, 634, 638, 640, 641, 651, 653, 654, 667, 689, 700, 715, 730, 736, or 737 in mouse eyes. Vitreous and retina samples were collected 15 minutes after eye drop application of IR800 labeled adalimumab-PC formulation (1:20 ratios) in Buffer 1 (10 ug in 3 uL per eye). Intraocular IR800-adalimumab signal±SEM, N=2 eyes.

FIG. 11 is a graph showing the results of intraocular penetration screening of IR800-secukinumab with peptide carrier eye drop formulations containing peptide carrier (OPC) 1, 498, 597, 608, 631, 633, 640, 653, 654, 689, 698, 700, 715, 730, 736, 737, 746, 747, 750, 759, 762, 764, 770, 776, or 777 in mouse eyes. Vitreous and retina samples were collected 15 minutes after eye drop application of IR800 labeled secukinumab-PC formulation (1:20 ratios) in Buffer 1 (10 ug in 3 uL per eye). Intraocular IR800-secukinumab signal±SEM, N=2 eyes.

FIG. 12 is a graph showing the results of intraocular penetration screening of IR800-guselkumab with peptide carrier eye drop formulations containing peptide carrier (OPC) 1, 498, 597, 608, 631, 633, 640, 654, 689, 698, 700, 703, 715, 730, 736, 737, 746, 747, 750, 759, 762, 764, 770, or 777 in mouse eyes. Vitreous and retina samples were collected 15 minutes after eye drop application of IR800 labeled guselkumab-PC formulation (1:20 ratios) in Buffer 2 (10 ug in 3 uL per eye). Intraocular IR800-guselkumab signal±SEM, N=2 eyes.

FIG. 13 is a graph showing the results of intraocular penetration screening of IR800-ustekinumab with peptide carrier eye drop formulations containing peptide carrier (OPC) 1, 498, 597, 608, 631, 633, 640, 653, 654, 689, 698, 700, 715, 730, 736, 737, 746, 747, 750, 759, 762, 764, 770, 776, or 777 in mouse eyes. Vitreous and retina samples were collected 15 minutes after eye drop application of IR800 labeled ustekinumab-PC formulation (1:20 ratios) in Buffer 1 (10 ug in 3 uL per eye). Intraocular IR800-ustekinumab signal±SEM, N=2 eyes.

FIG. 14 is a graph showing the results of intraocular penetration screening of IR800-pembrolizumab with peptide carrier eye drop formulations containing peptide carrier (OPC) 1, 498, 529, 564, 572, 578, 579, 597, 604, 608, 623, 631, 633, 638, 640, 641, 651, 653, 654, 658, 667, 670, 689, 700, 715, 730, 736, 737, or 750 in mouse eyes. Vitreous and retina samples were collected 15 minutes after eye drop application of IR800 labeled pembrolizumab-PC formulation (1:20 ratios) in Buffer 2 (10 ug in 3 uL per eye). Intraocular IR800-pembrolizumab signal±SEM, N=2 eyes.

FIG. 15 is a graph showing the results of intraocular penetration screening of IR800-nivolumab with peptide carrier eye drop formulation containing peptide carrier (OPC) 1, 498, 529, 564, 572, 578, 579, 597, 604, 608, 631, 633, 654, 667, 689, 698, 700, 703, 715, 730, 733, 736, 737, 746, 747, 750, 759, 762, or 777 in mouse eyes. Vitreous and retina samples were collected 15 minutes after eye drop application of IR800 labeled nivolumab-PC formulation (1:20 ratios) in Buffer 1 (10 ug in 3 uL per eye). Intraocular IR800-nivolumab signal±SEM, N=2 eyes.

FIG. 16 is a graph showing the results of intraocular penetration screening of IR800-ipilmumab with peptide carrier eye drop formulation containing peptide carrier (OPC) 1, 498, 572, 578, 579, 597, 604, 608, 631, 633, 654, 689, 700, 715, 730, 736, 737, 746, 747, 750, 759, 762, 770, or 777 in mouse eyes. Vitreous and retina samples were collected 15 minutes after eye drop application of IR800 labeled ipilimumab-PC formulation (1:20 ratios) in Buffer 2 (10 ug in 3 uL per eye). Intraocular IR800-ipilimumab signal±SEM, N=2 eyes.

FIGS. 17A-17E are graphs showing the results of intraocular penetration screening of recombinant proteins adiponectin (FIG. 17A), angiostatin (FIG. 17B), endostatin (FIG. 17C), GM-CSF (FIG. 17D), and IL-2 (FIG. 17E) with a peptide carrier eye drop formulation containing peptide carrier 498 in mouse eyes. Vitreous and retina samples were collected 15 minutes after eye drop application of IR800 labeled recombinant protein-PC formulation (1:20 ratios) in Buffer 1 (adiponectin, 2 ug; angiostatin, 1 ug; endostatin, 0.5 ug; GM-CSF, 2 ug; IL-2, 1 ug in 3 uL per eye). Intraocular IR800-protein signal±SEM, N=2 eyes.

FIG. 18 provides whole eyeball images of aflibercept biosimilar with peptide carrier (PC) formulations in mouse eyes. C57Bl/6J mouse eyeballs were cross-sectioned after 60 min post eye drop applications. Eye drop formulations of IR800-aflibercept (5 ug in 3-5 uL per eye) in Buffer 1 with each carrier were applied into mouse eyes. Arrows indicate the cornea.

FIGS. 19A-19B are graphs showing the concentration response of intraocular penetration efficiency of eye drop formulations in mouse eyes. Vitreous and retina samples were collected 15 minutes after eye drop application of IR800 labeled aflibercept-PC formulation (1:20 ratios) in Buffer 1 (5 ug in 3 uL per eye). FIG. 19A: Intraocular IR800-aflibercept/PC498; and FIG. 19B: intraocular IR800-aflibercept/PC654. IR680 and IR800 signal±SEM, N=4 eyes per point.

FIGS. 20A-20D are graphs illustrating data from a study characterizing molar ratios of antibody to peptide carrier for ocular penetration efficiency. Intraocular penetration activity was tested with different ratios of peptide carrier and payload peptide in mouse eyes. Vitreous and retina samples were collected 30 minutes after eye drop application of IR800 labeled antibody-PC formulation in Buffer 1. FIG. 20A: IR800-aflibercept (5 ug)/PC498; FIG. 20B: IR800-aflibercept (5 ug)/PC654; FIG. 20C: IR800-brolucizumab (3 ug)/PC498; and FIG. 20D: IR800-bevacizumab (10 ug)/PC654. IR800-antibody signal±SEM, N=2 eyes per point.

FIGS. 21A-21B are graphs showing the results of ELISA detection of antibody after applying eye drops to a mouse. Antibody:PC eye drop formulation was applied to the mouse 15 min before harvesting vitreous and retina. Diluted samples were applied to human Fc coated (1 ug/ml) ELISA plates. FIG. 21A: aflibercept (10 ug, 1:20 ratio) with P1 reference and PC654 in Buffer 1; FIG. 21B: PMC-403 anti-Tie-2 Ab (10 ug, 1:20 ratio) with PC572, PC578 and PC633 in Buffer 1 and Buffer 4 conditions. Intraocular Ab concentration in ng/ml±SEM, N=4 eyes per point.

FIGS. 22A-22B are graphs showing VEGF binding of IR800-aflibercept in eye drop formulation using ELISA. ELISA plates were coated with recombinant human VEGF (1 ug/ml) overnight at 4° C. Wells were blocked with 1% BSA with 0.05% Tween-20 (PBST) at RT before adding IR800-aflibercept/PC (5 ug/ml) complex binding to VEGF for 2 hrs. FIG. 22A: aflibercept with PC654 (square), PC715 (triangle) and PC537 (upside down triangle); FIG. 22B: aflibercept with PC747 (square), PC750 (triangle), PC751 (upside down triangle) and PC759 (diamond). Plates were washed 4× PBST and read in LiCor Odyssey CLx (VEGF bound aflibercept signal±SEM, N=2 per point).

FIGS. 23A-23B are graphs showing decreased levels of mVEGF following eye drop aflibercept-peptide carrier formulation administration to mouse eyes. Mouse VEGF (20 ng) was injected into the vitreous of C57Bl/6 mouse eyes (8-week old) before applying eye drops of aflibercept/PC formulation. FIG. 23A: Concentration response of aflibercept/PC381(1 ug, 5 ug and 15 ug/eye drop) in Buffer 1. After 40 minutes, vitreous and retina were collected to detect mVEGF levels in ELISA. FIG. 23B: mVEGF levels after administration of aflibercept (10 ug)/PC498 eye drops in Buffer 1. VEGF concentration±SEM, N=6 eyes per point.

FIG. 24 shows reduction of lesion area by administration of antibody-peptide carrier eye drop formulation in mouse eyes. Laser burned C57Bl/6J mouse eyes (female, 8-week, 4 laser burns/eye, total N=48 sites) were treated with either brolucizumab (5 ug)/PC633 or aflibercept (20 ug)/PC633 eye drops in Buffer 1 for 7 days (5 drops per day/eye). Choroid neovascular lesion was stained with Isolectin B4 conjugated with AF-647 (10 ug/ml) in flat mounted eyes and imaged using ECHO Revolve microscope. CNV lesion area±SEM, N=33-37 sites per condition.

FIGS. 25A-25C show a reduction of lesion area by aflibercept/PC633 in a mouse model of laser-induced choroid neovascular lesion. OCT image analysis was performed after eye drop treatment with aflibercept-PC633 formulation in mouse eyes. FIG. 25A: Laser induced photocoagulation (at 270 mW/80 ms, calibrated at 18.5 mW) was done by the Micron IV system in 8-week old C57Bl/6J mouse eyes. OCT images were acquired before and after eye drops for 7-day (5 drops/day). Arrows indicate laser induced lesion areas. FIG. 25B: Estimation of lesion area. FIG. 25C: Lesion volume changes before and after treatment using InSight software (Phoenix Technology). Statistical analysis was done by Graphpad Prism 7. Percent change of lesion area and lesion volume±SEM, N=30-38 sites per analysis.

FIGS. 26A-26E arise from a study characterizing the reduction of vascular leakiness following topical administration of aflibercept/PC654 formulation in mouse eyes. FIG. 26A: Representative Fundus fluorescein angiography (FFA) images; FFA scan was performed at 7-day and 14-day post laser burn in C57Bl/6J mouse eyes (female, 8-week, 4 laser burns/eye) by injecting sodium fluorescein (100 ug, IV) and randomized by eyes with unhealed active lesion sites before eye drop treatment with aflibercept (20 ug)/peptide carrier 654 in Buffer 1 from Day-8 (5 drops per day/eye for 7 days). FIG. 26B: FFA intensity changes were compared before and after eye drop treatment (N=15-27 sites per condition). FIGS. 26C and 26D: FFA intensity changes by intravitreal injection of Eylea® (20 ug/eye) in Buffer 2 as a comparison (N=33-39 sites per condition). Data are FFA intensity±SEM, N=15-39. FIG. 26E: schematic of the study protocol.

FIGS. 27A-27C arise from a study characterizing intraocular penetration screening of IR800-aflibercept/PC formulation in pig eyes. FIG. 27A: IR800 labeled aflibercept alone (50 ug/50 uL, 2 drops per eye) or IR800-aflibercept with PC631 formulation (1:20 ratios, 50 ug/50 uL, 2 drops per eye in Buffer 1) in Yorkshire pig eyes (12-week, N=2 eyes per condition) were administered as eye drops over 2 hrs. Enucleated eyes were fixed in 4% PFA and harvested retina, vitreous humor and lens were harvested. FIG. 27B: Intraocular IR800-aflibercept intensity was scanned in LiCor Odyssey, and data were plotted for vitreous humor and retina (Signal±SEM, N=2 eyes per condition). FIG. 27C: schematic of the study protocol.

FIGS. 28A and 28B show the results of a study characterizing intraocular penetration of single or dual antibody/PC eye drops into mouse eyes. IR800-aflibercept and IR680-PMC401s (anti-Ang-2 Ab) were formulated with PC654 (FIG. 28A) or PC747 (FIG. 28B) in Buffer 1, and applied as a single antibody eye drop or combined for the dual antibody eye drop into mouse eyes for 15 minutes. Mouse vitreous and retina were harvested after enucleation and detected on SDS-PAGE gels. Dye conjugated Abs were detected by LiCor Odyssey.

### DETAILED DESCRIPTION

The present disclosure is based, at least in part, on the discovery of peptides that are capable of delivering large molecules including growth factors and therapeutic antibodies into the intraocular space (including aqueous chamber, vitreous, choroid and retina) of a subject without direct injection into the eye. The use of such peptides allows topical administration, which avoids injection-related issues including infection, pain, bleeding, inflammation and tissue damage.

### Peptide Carriers

In one aspect, described herein is a peptide (also referenced herein as “peptide carrier”) comprising no more than 50 amino acids in length and that comprises (or consists of) the amino acid sequence of Formula (1): X1-X2-X3-X4-X5-X6-X7-X8-X9-X10-X11-X12-X13-X14-X15-X16-X17-F-L-I/V-P-K-G, wherein
X1 is V, M, L, I, or K;
X2 is K, R, or H;
X3 is K, R, or H;
X4 is R, K, or H;
X5 is K, R, or H;
X6 is I, V, or L;
X7 is V, L, or I;
X8 is R, K, or H;
X9 is L, K, V, or R;
X10 is I, L, or V;
X11 is K, R, or H;
X12 is F, Y, K, or L, or is absent;
X13 is L, V, or I, or is absent;
X14 is V, L, or I, or is absent;
X15 is K, L, R, or H, or is absent;
X16 is (i) M, L, V, I, R, A, K, G, C, Y, S, N, or D; is an amino acid of (i) in combination with L, K, R, or V, or (iii) is absent; and
X17 is F, Y, FL, YL, FLK, YLK, FLKL, or YLKL.

In some aspects, the peptide comprises at least 15 amino acids and is less than 50 amino acids in length. In some aspects, the peptide comprises at least 20 amino acids and is less than 50 amino acids in length. In some aspects, the peptide is 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 39, or 50 amino acids.

In some aspects, the peptide comprises (or consists of) the amino acid sequence of Formula (2): X1-X2-X3-X4-X5-X6-X7-X8-X9-X10-X11-X12-X13-X14-X15-X16-X17-F-L-I-P-K-G.

In some aspects, the peptide comprises (or consists of) the amino acid sequence of Formula (3): L/V-K-K-R-K-I/V-V-R-L-I-K-X12-X13-X14-X15-X16-F-F-L-I-P-K-G, wherein
X12 is F or Y or is absent;
X13 is L or is absent;
X14 is L or V or is absent;
X15 is K or is absent; and
X16 is L or M or is absent.

In some aspects, the peptide further comprises the amino acid sequence of one of SEQ ID NOs: 103-124 [Z1-Z23, provided below in Table A], attached at the C-terminus.

TABLE A

| SEQ ID NO. | | |
|---|---|---|
| 103 | Z1 | RRRRRR |
| 104 | Z2 | RRRR |
| 105 | Z3 | RYLE |
| 106 | Z4 | RPARPAR |
| 107 | Z5 | MASIWVGHRGAVPF |
| 108 | Z6 | RPSRPRIRYKC |
| 109 | Z7 | RRKRRR |
| 110 | Z8 | ARPCA |
| 111 | Z9 | GVITRIR |
| 112 | Z10 | ACSSSPSKHCG |
| 113 | Z11 | LVGVFH |
| — | Z12 | E/LE |
| 114 | Z13 | HAIYPRH |
| 115 | Z14 | GYRPVHNIRGHWAPG |
| 116 | Z15 | AHLHNRS |
| 117 | Z16 | NAHQARST |
| 118 | Z17 | QHREDGS |
| 119 | Z18 | NLLMAAS |
| 120 | Z19 | DKPRR |
| 121 | Z20 | RPAPR |
| 122 | Z21 | CNGRCG |
| 123 | Z22 | THRPPMWSPVWP |
| 124 | Z23 | RPLKPW |

In some aspects, the peptide further comprises, attached at the N-terminus, L, L-L, or the amino acid sequence X30-V/L-G-V/L-F-H (Formula (4)), wherein X30 is L, I, or V. In some aspects, the amino acid sequence attached to the N-terminus is L-V-G-V-F-H (SEQ ID NO: 102). The notation of “amino acid 1/amino acid 2” in the formulas described herein denotes that amino acid 1 and amino acid 2 are alternatives at the recited position. For instance, “V/L” denotes that V or L may be present at the recited position.

In some aspects, the peptide comprises (or consists of) the amino acid sequence set forth in any one of SEQ ID NOs: 1-72. In some aspects, the peptide comprises the amino acid sequence set forth in any one of SEQ ID NOs: 20, 21, 24, 30, 53, 60, 67, and 72. In some aspects, the peptide comprises the amino acid sequence set forth in any one of SEQ ID NOs: 1-40. In some aspects, the peptide comprises the amino acid sequence set forth in any one of SEQ ID NOs: 41-59. In some aspects, the peptide comprises the amino acid sequence set forth in any one of SEQ ID NOs: 60-72. Representative peptides of the disclosure are provided in Tables 1-3 below.

TABLE 1
Group 1 Peptides

| | SEQ | |
|---|---|---|
| Peptide ID | ID | |
| (“OPC”) | NO: | SEQUENCE |
| 712 | 1 | LKKRKVVRLI KFLLKFFLIPKG LVGVFH |
| 715 | 2 | LKKRKVVRLI KFLLKMFFLIPKG LVGVFH |
| 721 | 3 | IKKRKVVRLI KFLLKLFFLIPKG LVGVFH |
| 722 | 4 | VKKRKVVRLI KFLLKLFFLIPKG LVGVFH |
| 723 | 5 | MKKRKVVRLI KFLLKLFFLIPKG LVGVFH |
| 724 | 6 | MKKRKVVRLI KYLLKLFFLIPKG LVGVFH |
| 725 | 7 | IKRRKVVRLI KFLLKLFFLIPKG LVGVFH |
| 726 | 8 | IKKRKVVRLI KYLLKLFFLIPKG LVGVFH |
| 728 | 9 | IKKRKVVRLI KKLLKLFFLIPKG LVGVFH |
| 729 | 10 | IKKRKVLRLI KYLLKLFFLIPKG LVGVFH |
| 730 | 11 | IKKRKVLRLI KYLVKLFFLIPKG LVGVFH |
| 731 | 12 | IKKRKIIRLI KFLLKLFFLIPKG LVGVFH |
| 733 | 13 | IKKRKIVRLI KFLVKLFFLIPKG LVGVFH |
| 735 | 14 | LKKRKVVRLI KFLLFFLIPKG LVGVFH |
| 736 | 15 | LKKRKVVRLI KFLFFLIPKG LVGVFH |
| 737 | 16 | LKKRKVVRLI KFFFLIPKG LVGVFH |
| 742 | 17 | LKKRKVVRLI KFLLKLLVGVFHFFLIPKG |
| 743 | 18 | FFLIPKG LKKRKVVRLI KFLLKLLVGVFH |
| 745 | 19 | VKKRKIVRLI KFLVKLFFLIPKG LVGVFH |
| 746 | 20 | VKKRKIVRLI KFLVKMFFLIPKG LVGVFH |
| 747 | 21 | VKKRKIVRLI KFLLKFFLIPKG LVGVFH |
| 748 | 22 | VKKRKVVRLI KFLLKFFLIPKG LVGVFH |
| 749 | 23 | VKKRKVVRLI KLFFLIPKG LVGVFH |
| 750 | 24 | VKKRKVVRLI KYFFLIPKG LVGVFH |
| 751 | 25 | VKKRKIVRLI KVFFLIPKG LVGVFH |
| 752 | 26 | VKKRKVVRLI KFVLKFFLIPKG LVGVFH |
| 753 | 27 | VKKRKVVRLI KFVLKYFLIPKG LVGVFH |
| 754 | 28 | VKKRKLVRLI KFVLKFFLIPKG LVGVFH |
| 755 | 29 | VKKRKIVRLI KFVLKFFLIPKG LVGVFH |
| 759 | 30 | VKKRKIVRLI KFFLIPKG LVGVFH |
| 760 | 31 | VKKRKIVRLI FFLIPKG LVGVFH |
| 761 | 32 | VKKRKIVRLI KFLLKFFLIPKG IVGVFH |
| 762 | 33 | VKKRKIVRLI KFLLKYFLIPKG VVGVFH |
| 763 | 34 | VKKRKVVRLI KYFFLIPKG VVGVFH |
| 764 | 35 | VKKRKIVRLI KYFFLIPKG VVGVFH |
| 765 | 36 | VKKRKIVRLI KYFFLVPKG IVGVFH |
| 766 | 37 | VKKRKVVRLI KYYFLIPKG IVGVFH |
| 775 | 38 | VKKRKVIRLI KFFLIPKG LVGVFH |
| 776 | 39 | VKKRKILRLI KFFLIPKG LVGVFH |
| 777 | 40 | VKKRKIVRLI KFFLIPK LVGVFH |

TABLE 2
Group 2 Peptides

| Peptide ID | SEQ ID | |
|---|---|---|
| (“OPC”) | NO: | SEQUENCE |
| 381 | 41 | LKKRKVVRLI KFLLKLLKKR KVVFFLIPKG RRRRRR |
| 498 | 42 | LKKRKVVRLI KFLLKLLKKR KVVFFLIPKG RRRR |
| 596 | 43 | LKKRKVVRLI KELLKL FFLIPKG RRRR |
| 597 | 44 | LKKRKVVRLI KFLLKLLKK FFLIPKG RRRR |
| 608 | 45 | LKKRKVVRLI KFLLKLLKKFFLIPKG ACSSSPSKHCG |
| 633 | 46 | LKKRKVVRLI KELLKL FFLIPKG RPARPAR |
| 638 | 47 | LKKRKVVRLI KFLLKLFFLIPKG THRPPMWSPVWP |
| 640 | 48 | LKKRKVVRLI KFLLKLFFLIPKG GYRPVHNIRGHWAPG |
| 641 | 49 | LKKRKVVRLI KFLLKFFLIPKG RLYE |
| 642 | 50 | LKKRKVVRLI KFLLKFFLIPKG RPARPAR |
| 651 | 51 | LKKRKVVRLI KFLLKLFFLIPKG AHLHNRS |
| 653 | 52 | LKKRKVVRLI KFLLKLFFLIPKG NAHQARST |
| 654 | 53 | LKKRKVVRLI KFLLKLFFLIPKG LVGVFH |
| 656 | 54 | LKKRKVVRLI KFLLKLFFLIPKG QHREDGS |
| 658 | 55 | LKKRKVVRLI KFLLKLFFLIPKG RPLKPW |
| 664 | 56 | LKKRKVVRLI KFLLKLLVFFLIPKG RLYE |
| 698 | 57 | LKKRKVVRLI KFLLKLFFLIPKG NLLMAAS |
| 700 | 58 | LKKRKVVRLI KELLKLFFLIPKG DKPRR |
| 703 | 59 | LKKRKVVRLI KFLLKLFFLIPKG RPAR |

TABLE 3
Group 3 peptides

| | SEQ | |
|---|---|---|
| Peptide ID | ID | |
| (“OPC”) | NO: | SEQUENCE |
| 689 | 60 | LVGVFH LKKRKVVRLI KFLLKLFFLIPKG |
| 738 | 61 | LVGVFH LKKRKVVRLI KFLLKFFLIPKG |
| 739 | 62 | LVGVFH LKKRKVVRLI KFLLFFLIPKG |
| 740 | 63 | LVGVFH LKKRKVVRLI KFLFFLIPKG |
| 741 | 64 | LVGVFH LKKRKVVRLI KFFFLIPKG |
| 768 | 65 | LVGVFH VKKRKIVRLI KFLVKFFLIPKG |
| 769 | 66 | IVGVFH VKKRKIVRLI KFLVKFFLIPKG |
| 770 | 67 | LVGVFH VKKRKIVRLI KFFFLIPKG |
| 771 | 68 | IVGVFH VKKRKIVRLI KFYFLIPKG |
| 772 | 69 | IVGVFH VKKRKIVRLV KFYFLIPKG |
| 773 | 70 | LVGVFH VKKRKVVRVI KFFFLIPKG |
| 788 | 71 | VVGVFH VKKRKIVRLI KFLLKFFLIPKG |
| 789 | 72 | VVGVFH VKKRKIVRLI KFFFLIPKG |

The disclosure also provides a peptide comprising the amino acid sequence of any one of SEQ ID NOs: 1-72 comprising one, two, or three substitutions within the amino acid sequence of SEQ ID NOs: 1-72. For instance, peptides comprising the amino acid sequence of peptide carriers 721, 722, 723, and 724 are substitutional mutants of the amino acid sequence of peptide carrier 712. Optionally, the substitutions are conservative substitutions. In various aspects, the substitutions are selected from the following options, wherein the amino acid positions correspond to the positions set forth in Formula (1):
X1 is V, M, L, I, or K;
X2 is K, R, or H;
X3 is K, R, or H;
X4 is R, K, or H;
X5 is K, R, or H;
X6 is I, V, or L;
X7 is V, L, or I;
X8 is R, K, or H;
X9 is L, K, V, or R;
X10 is I, L, or V;
X11 is K, R, or H;
X12 is F, Y, K, or L;
X13 is L, V, or I;
X14 is V, L, or I;
X15 is K, L, R, or H;
X16 is (i) M, L, V, I, R, A, K, G, C, Y, S, N, or D; is an amino acid of (i) in combination with L, K, R, or V; and
X17 is F, Y, FL, YL, FLK, YLK, FLKL, or YLKL.

The disclosure also provides a peptide comprising the amino acid sequence of any one of SEQ ID NOs: 73-98 comprising one, two, or three substitutions within the amino acid sequence of SEQ ID NOs: 73-98. A peptide comprising any one of SEQ ID NOs: 73-98 is also contemplated. The disclosure further provides a peptide comprising the amino acid sequence of any one of SEQ ID NOs: 125-128 comprising one, two, or three substitutions within the amino acid sequence of SEQ ID NOs: 125-128. A peptide comprising any one of SEQ ID NOs: 125-128 is also contemplated.

TABLE 4

| Peptide ID | SEQ ID | |
|---|---|---|
| (“OPC”) | NO: | SEQUENCE |
| 1 | 73 | RRRRRR |
| 9 | 74 | RKKRRQRRR |
| 499 | 75 | KKLFKKILKYLKKLFKKILKYL FFLIPKG RRRRRR |
| 502 | 76 | LKKRKVVRLI KELLKLLKKR KVVFFLIPKG ACSSSPSKHCG |
| 514 | 77 | KKLFKKILKY LINLKALAAL AKKIL RPSRPRIRYKC |
| 529 | 78 | LKKRKVVRLI KFLLKLLKKR KVVFFLIPKG RRRRRR AVPF |
| 564 | 79 | LKKRKVVRLI KFLLKLLKKR KVVFFLIPKG MASIWVGHRG AVPF |
| 571 | 80 | LKKRKVVRLI KFLLKLLKKR KVVFFLIPKG RRRR RLYE |
| 572 | 81 | LKKRKVVRLI KELLKLLKKR KVVFFLIPKG RLYE |
| 578 | 82 | KKLFKKILKY LKKLFKKILK YLRPSRPRIRYKC RPARPAR |
| 579 | 83 | LKKRKVVRLI KELLKLLKKR KVVFFLIPKGRRRRPARPAR |
| 587 | 84 | LKKRKVVRLI KFLLKLLKKR KVVFFLIPKG RRRR RRKRRR |
| 591 | 85 | LKKRKVVRLI KFLLKLLKKR KFFLIPKG ACSSSPSKHCG |
| | | RRKRRR |
| 595 | 86 | LKKRKVVRLI KFLLKLLKKR KFFLIPKG ACSSSPSKHCG |
| 603 | 87 | LKKRKVVRLI KNLLKLLKKR KVNFFLIPKG RRRR |
| 604 | 88 | LKKRKVVRLI KFNLKLLKKR KVNFFLIPKG RRRR |
| 607 | 89 | LKKRKVVRLI KFLLKLFFLIPKG ACSSSPSKHCG |
| 622 | 90 | KKRKVVRLI KELLKLLKKR KVVFFLIPKG |
| 623 | 91 | LKKRKVVRLI KFLLKLLKKR KVVFFLIPKG E |
| 624 | 92 | LKKRKVVRLI KFLLKLLKKR KVVFFLIPKG LE |
| 631 | 93 | LKKRKVVRLI KFLLKLFFLIPKG RLYE |
| 634 | 94 | LKKRKVVRLI KELLKLFFLIPKG HHIYLGAVNYIY |
| 648 | 95 | LKKRKVVRLI KFLLKLLKKFFLIPKG LE |
| 667 | 96 | LKKRKVVRLI KFLLKLRFFLIPKG RLYE |
| 670 | 97 | KAFDITYVRLKFFLIPKG |
| 744 | 98 | FFLIPKG LVGVFH LKKRKVVRLI KFLLKL |
| 685 | 125 | KAFDITYVRL KFFLIPKGRP ARPAR |
| 798 | 126 | VKKRKIVRLI KFFLIPKLVG VFHRLYE |
| 808 | 127 | KKLKKLKVRL VKFLLKFGPL RYRVVLFIER IKLK |
| 825 | 128 | KKLKKLKVRL VKFLLKFGPL RYRVVL |

In some aspects, the peptides described herein are chemically modified. Exemplary modifications include, but are not limited to, acetylation, lipidation (e.g., palmitoylation, myristoylation, prenylation or glycosyl phosphatidylinositol anchor), pegylation, methylation, amidation, cyclization, L to D-amino acid conversion, glycosylation, sulfation, hydroxylation, phosphorylation, ubiquitination, isomerization, flavin binding, pyrrolidone binding or nitrosylation. The disclosure contemplates a peptide disclosed herein further comprising a functional group that facilitates conjugation to another moiety (e.g., a peptide moiety). Exemplary functional groups include, but are not limited to, isothiocyanate, isocyanate, acyl azide, NHS ester, sulfonyl chloride, aldehyde, epoxide, oxirane, carbonate, arylating agent, imidoester, carbodiimide, anhydride, alkyl halide derivatives (e.g., haloacetyl derivatives), maleimide, aziridine, acryloyl derivatives, arylating agents, thiol-disulfide exchange reagents (e.g., pyridyl disulfides or TNB thiol), diazoalkane, carboyldiimadazole, N,N′-Disuccinyl carbonate, N-Hydroxysuccinimidyl chloroformate, and hydrazine derivatives.

The disclosure also provides a nucleic acid comprising a nucleic acid sequence encoding a peptide described herein (or a construct described below). Methods of preparing DNA and/or RNA molecules are known in the art. In one aspect, a DNA/RNA molecule encoding a peptide provided herein is generated using chemical synthesis techniques and/or using polymerase chain reaction (PCR). If desired, a peptide coding sequence is incorporated into an expression vector. One of ordinary skill in the art will appreciate that any of a number of expression vectors known in the art are suitable in the context of the disclosure, such as, but not limited to, plasmids, plasmid-liposome complexes, and viral vectors. Any of these expression vectors may be prepared using standard recombinant DNA techniques described in, e.g., Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994). Optionally, the nucleic acid is operably linked to one or more regulatory sequences, such as a promoter, activator, enhancer, cap signal, polyadenylation signal, or other signal involved with the control of transcription or translation.

### Constructs

In another aspect, the disclosure provides a construct comprising a peptide carrier (also referred to herein as “PC” or “OPC”) described herein conjugated (i.e., attached) to one or more payload peptides.

A “payload peptide” may be any peptide of interest suitable for in vivo delivery, such as delivery to the eye. In some aspects, the payload peptide mediates a biological effect in vivo, such as biological effect in the eye. Examples of payload peptides include, but are not limited to, anti-angiogenic agents, neuroprotective agents, anti-infective agents, growth factors, growth factor antagonists, cytokines, and anti-inflammatory agents. For instance, contemplated payload peptides include, e.g., VEGF decoys, endostatin, collagen, nerve growth factor (NGF), angiostatin, plasminogen, angiopoietin, glial cell line-derived neurotrophic factor (GDNF), peripherin-2, RPE65 (retinoid isomerase), retinitis pigmentosa GTPase regulator (RPGR), adiponectin, granulocyte-macrophage colony-stimulating factor (GM-CSF), IL-2, antitrypsin, calcitonin, cadherin, angiopoietin-1 derived peptide, SNARE binding peptide, collagen binding peptide, (Col4 binding peptide), transferrin receptor binding peptide (TfR BP), mastofaran, surfactant, FasL inhibitor peptide, annexin A, hemagglutinin binding peptide, VEGFR2 binding antagonist peptide, somatostatin, dynamin inhibitor peptide, beta selectin binding peptide, aquaporin binding peptide, RGD, neuropilin binding peptide, follistatin, SPARC, thrombospondin-1, thymosin beta4, integrin avb binding peptide, semaphoring-7 binding peptide, and Tie-2 inhibitor peptide. In some aspects, the payload peptide is a fusion protein. Exemplary fusion proteins include, but are not limited to, aflibercept, asunercept, and etanercept.

In some aspects, the payload peptide is an antibody, or comprises an antigen-binding antibody fragment, or is an antibody-like construct. The term “antibody” refers to an intact antigen-binding immunoglobulin. The antibody can be an IgA, IgD, IgE, IgG, or IgM antibody, including any one of IgG1, IgG2, IgG3, or IgG4. In various embodiments, an intact antibody comprises two full-length heavy chains and two full-length light chains. Antigen-binding antibody fragments include, but are not limited to, Fab′, Fab, F(ab′)2, and Fv fragments. The architecture of antibodies has been exploited to create a growing range of alternative formats that span a molecular-weight range of at least about 12-150 kDa and have a valency (n) range from monomeric, to dimeric, to trimeric, to tetrameric, and potentially higher; such alternative formats are referred to herein as “antibody-like constructs.” Antibody-like constructs include those based on the full antibody structure and those that mimic antibody fragments which retain full antigen-binding capacity, e.g., scFvs and VHH. Antibody-like constructs may comprise antigen-binding fragments of antibodies.

Exemplary antibodies or fragments thereof include, but are not limited to, ranibizumab, brolucizumab, bevacizumab, faricimab, ramucirumab, fresolimumab, rcetuximab, panitumumab, adalimumab, infliximab, brodalumab, ixekizumab, secukinumab, ustekinumab, guselkumab, canakinumab, toclizumab, sarilumab, nivolumab, olinvacimab, atezolizumab and ipilimumab. Exemplary antibodies or fragments thereof include, but are not limited to, antibodies or fragments thereof that bind angiopoietin-2, VEGFR2, tumor growth factor-beta (TGFb), or Tie-2 (Angiopoietin-1 receptor, Tyrosine-protein kinase receptor TEK).

In some aspects, the construct comprises a peptide carrier comprising the amino acid sequence set forth in any one of SEQ ID NOs: 1-40 and one or more payload peptides, such as aflibercept, ranibizumab, brolucizumab, bevacizumab, faricimab, ramucirumab, fresolimumab, cetuximab, panitumumab, asunercept, PMC-401 (anti-angiopoietin-2 IgG antibody; PharmAbscine), or PMC-401s (scFv Ang-2 antibody; PharmAbscine). In some aspects, the construct comprises a peptide carrier comprising the amino acid sequence set forth in SEQ ID NO: 2, 16, 21, 24, 25, or 30 and the payload peptide is aflibercept.

In some aspects, the construct comprises a peptide carrier comprising the amino acid sequence set forth in any one of SEQ ID NOs: 1-40 and two or more payload peptides, wherein the payload peptides are optionally selected from aflibercept, ranibizumab, brolucizumab, bevacizumab, faricimab, ramucirumab, fresolimumab, cetuximab, panitumumab, asunercept, and PMC401. In some aspects, the construct comprises a peptide carrier comprising the amino acid sequence set forth in any one of SEQ ID NOs: 1-40 and two or more payload peptides, wherein the two or more payload peptides are aflibercept and PMC-401. In some aspects, the construct comprises a peptide carrier comprising the amino acid sequence set forth in SEQ ID NO: 21 and the payload peptides are aflibercept and PMC-401.

In some aspects, the construct comprises a peptide carrier comprising the amino acid sequence set forth in any one of SEQ ID NOs: 41-59 and one or more payload peptides, such as adalimumab, infliximab, etanercept, brodalumab, ixekizumab, secukinumab, ustekinumab, guselkumab, canakinumab, toclizumab, sarilumab, RPE65, RPGR, nivolumab, atezolizumab, ipilimumab or tebentafusp. In some aspects, the construct comprises a peptide carrier comprising the amino acid sequence set forth in SEQ ID NO: 42 and the payload peptide is aflibercept. In some aspects, the construct comprises a peptide carrier comprising the amino acid sequence set forth in SEQ ID NO: 53 and the payload peptide is aflibercept or bevacizumab. In some aspects, the construct comprises a peptide carrier comprising the amino acid sequence set forth in SEQ ID NO: 46 and the payload peptide is brolucizumab or aflibercept.

In some aspects, the construct comprises a peptide carrier comprising the amino acid sequence set forth in any one of SEQ ID NOs: 41-59 and two or more payload peptides, wherein the payload peptides are optionally selected from adalimumab, infliximab, etanercept, brodalumab, ixekizumab, secukinumab, ustekinumab, guselkumab, canakinumab, toclizumab, sarilumab, RPE65, RPGR, nivolumab, atezolizumab, ipilimumab and tebentafusp.

In some aspects, the construct comprises a peptide carrier comprising the amino acid sequence set forth in any one of SEQ ID NOs: 60-72 and one or more payload peptides, such as adalimumab, infliximab, etanercept, brodalumab, ixekizumab, secukinumab, ustekinumab, guselkumab, canakinumab, toclizumab, sarilumab, RPE65, RPGR, nivolumab, atezolizumab, ipilimumab, tebentafusp, aflibercept, ranibizumab, brolucizumab, bevacizumab, faricimab, ramucirumab, fresolimumab, and asunercept.

In some aspects, the construct comprises a peptide carrier comprising the amino acid sequence set forth in any one of SEQ ID NOs: 60-72 and two or more payload peptides, wherein the payload peptides are optionally selected from adalimumab, infliximab, etanercept, brodalumab, ixekizumab, secukinumab, ustekinumab, guselkumab, canakinumab, toclizumab, sarilumab, RPE65, RPGR, nivolumab, atezolizumab, ipilimumab, tebentafusp, aflibercept, ranibizumab, brolucizumab, bevacizumab, faricimab, ramucirumab, fresolimumab, and asunercept.

The peptide carrier may be conjugated (e.g., covalently attached) either directly to the payload peptide or via a linker. Representative linkers include, but are not limited to, glycine-serine polymers. Linkers include, but not are limited to, (Gly)n-Ser linkers (e.g., [(G4)S], [(G4)S]2, and [(G4)S]3); glycine linkers (e.g., (Gly)6); (Gly)n-Glu linkers (e.g., [(G4)E], [(G4)E]2, [(G4)E]3, [(G4)E](G4), and [(G4)E]2(G4)), (Gly)n-Gln linkers (e.g., [(G4)Q], [(G4)Q]2, [(G4)Q]3, [(G4)Q](G4), [(G4)Q]2(G4), and [(G4)Q]3(G4)), and Glu-Ala-based linkers (e.g., [EAAAK]3 and [EAAAK]4). In some aspects, the linker is a cleavable linker (e.g., cleavable with protease sensitive peptides (e.g., MMP2, MMP9, plasmin, tPA, Kallikrein)) or disulfide (S-S) in the fusion site. The payload peptide may be attached at either the N- or C-terminus of the peptide carrier.

Further, more than one peptide carrier may be used. In this regard, the construct may comprise, one, two, three, four, five, six, seven, or eight peptide carrier sequences, which may be the same or different. In various aspects, the payload peptide is indirectly attached to the peptide carrier via a cleavable (e.g., chemically or enzymatically cleavable) or non-cleavable linker. The linker may be any length, e.g., 1-60 amino acids, such as 1-5, 1-10, 2-18, 4-30, 5-15, or 10-25 amino acids, in length.

### Compositions

Compositions are provided which comprise the peptide carrier described herein, one or more peptide carriers and one or more payload peptides (e.g., an antibody, an antibody fragment, or a recombinant protein), or a construct comprising one or more peptide carriers described herein conjugated to one or more payload peptides, and one or more pharmaceutically acceptable carriers, diluents, or excipients. Optionally, the composition is an ophthalmic formulation, such as a composition suitable for topical administration to the ocular surface (e.g., an eye drop, eye ointment, or eye wash). The composition may be impregnated into a corneal shield or a contact lens or other wearable to provide direct contact to the ocular surface.

Optionally, the composition comprises payload peptide and peptide carrier in a molar ratio of the payload peptide to the peptide carrier from about 1:1 to about 1:70 (or about 1:1, 1:2, 1:3, 1:4, 1:5, 1:6, 1:7, 1:8, 1:9, 1:10, 1:15, 1:20, 1:25, 1:30, 1:35, 1:40, 1:45, 1:50, 1:55, 1:60, 1:65 or 1:70). In some aspects, the composition comprises payload peptide and peptide carrier in a molar ratio of the payload peptide to peptide carrier from about 1:1 to about 1:20 (e.g., from about 1:1 to about 1:5 or from about 1:5 to about 1:10) or from about 1:10 to about 1:20 (e.g., from about 1:10 to about 1:15 or from about 1:15 to about 1:20). Optionally, the composition comprises payload peptide and peptide carrier in a molar ratio of the payload peptide to peptide carrier from about 1:20 to about 1:60 (e.g., from about 1:20 to about 1:50, from about 1:20 to about 1:40, from about 1:35 to about 1:60, or from about 1:20 to about 1:35). In some aspects, the composition comprises payload peptide and peptide carrier in a molar ratio of the payload peptide to peptide carrier from about 1:1 to about 1:5, such as about 1:1, 1:2, 1:3, or 1:4.

In various aspects, the payload peptide present in the composition is aflibercept, ranibizumab, brolucizumab, bevacizumab, faricimab, ramucirumab, fresolimumab, cetuximab, panitumumab, asunercept, recCD59, NGM621 or ANX007, and the peptide carrier optionally comprises the amino acid sequence set forth in any one of SEQ ID NOs: 1-40. In various aspects, the payload peptide is adalimumab, infliximab, etanercept, brodalumab, ixekizumab, secukinumab, ustekinumab, guselkumab, canakinumab, toclizumab, sarilumab, RPE65, RPGR, nivolumab, atezolizumab, ipilimumab or ortebentafusp, and the peptide carrier optionally comprises the amino acid sequence set forth in any one of SEQ ID NOs: 41-59. In any of the aspects, the payload peptide is optionally conjugated to a drug. If desired, the composition is formulated as an eye drop.

Carriers and excipients that can be used to make pharmaceutical formulations (including topical formulations for ocular delivery) include without limitation solvents (e.g., aqueous solvents such as water, saline and PBS), isotonic/iso-osmotic agents (e.g., NaCl), buffers (e.g., acetate, ascorbate, borate, hydrogen carbonate/carbonate, citrate, gluconate, lactate, phosphate (such as sodium dihydrogen phosphate and disodium hydrogen phosphate), propionate and TRIS (tromethamine) buffers), and non-ionic surfactants (e.g., polysorbates, such as polysorbate 20). Additional substances suitable for inclusion in a pharmaceutically-acceptable formulation (including a formulation for topical delivery to the eye) include, e.g., hydrophilic or polar amino acids (e.g., arginine and histidine), polyols (e.g., mannitol and sorbitol), saccharides (e.g., glucose, lactose, sucrose and trehalose), and solubilizers (e.g., tyloxapol, fatty acid glycerol poly-lower alkylene glycol esters, fatty acid poly-lower alkylene glycol esters, polyethylene glycols or glycerol ethers).

A pharmaceutical composition may comprise one or more chemical penetration enhancers (CPEs) that enhance penetration of a macromolecule (e.g., a polypeptide) or a small molecule across tissue barriers or cell membranes. Exemplary CPEs include, but are not limited to, hydrocarbons (e.g., alkanes and alkenes, such as squalene); terpenes and terpenoids (e.g., D-limonene, carvone, eucalyptol, menthol, menthone and nerolidol); essential/volatile oils (e.g., anise oil, caraway oil, cardamom oil, chenopodium oil, eucalyptus oil and lemon oil); ethers and fatty ethers (e.g., 2-n-nonyl-1,3-dioxolane); phenols (e.g., eugenol); alcohols and fatty alcohols (e.g., methanol, ethanol, isopropyl alcohol, pentanol, lauryl alcohol, oleyl alcohol, benzyl alcohol, diethylene glycol mono-ethyl ether, propylene glycol, dipropylene glycol, polyethylene glycol and glycerol); benzoic acids (e.g., salicylic acid and acetylsalicylic acid); fatty acids (e.g., capric acid, lauric acid, myristic acid, oleic acid, linoleic acid and linolenic acid); esters, fatty alcohol esters and fatty acid esters (e.g., ethyl acetate, methyl laurate, isopropyl myristate, isopropyl palmitate, methyl oleate, ethyl oleate, propylene glycol mono-oleate, glycerol mono-oleate, triacetin and pentadecalactone); hydroxyl-containing esters, fatty alcohol esters and fatty acid esters (e.g., lauryl lactate, glyceryl/glycerol monolaurate, glycerol monoleate, sorbitan oleate and octyl salicylate); amines (e.g., diethanolamine and triethanolamine); amides, fatty amine amides and fatty acid amides (e.g., urea, dimethylformamide, dimethylacetamide, diethylacetamide, diethyltoluamide, N-lauroyl sarcosine, 1-dodecylazacycloheptane-2-one (laurocapram or AZONE®) and pyrrolidone compounds (e.g., 2-pyrrolidone and N-methyl-2-pyrrolidone)); ionic and non-ionic surfactants (e.g., cetyltrimethylammonium bromide, sodium laurate, sodium laureth sulfate, sodium cholate, sodium lauroyl sarcosinate, N-lauroyl sarcosine, sorbitan monolaurate, Brij® surfactants, Pluronic® surfactants, Tween® surfactants, saponins, alkyl glycosides, and fatty ether and fatty ester saccharides); phospholipids (e.g., lecithin); organic sulfoxides (e.g., dimethyl sulfoxide and decylmethyl sulfoxide); and ginsenoside.

### Methods

Methods of delivering a payload peptide to an eye of a subject are also contemplated. In some aspects, the method comprises administering a composition comprising one or more peptide carriers and one or more payload peptides directly to the eye of the subject, e.g., topically administered to the eye of the subject. Optionally, the method comprises administering a composition comprising a construct described herein (e.g., a construct comprising the peptide carrier conjugated to a payload peptide) directly to the eye of the subject, e.g., topically administered to the eye of the subject.

Also provided are methods of treating an ocular disorder in a subject in need thereof. In various aspects, the method comprises administering a composition comprising one or more peptide carriers and one or more payload peptides directly to the eye of the subject. For instance, the disclosure contemplates administering a composition comprising a construct described herein (e.g., a construct comprising a peptide carrier described herein conjugated to a payload peptide) directly to the eye of the subject (e.g., topically administered to the eye of the subject). Exemplary ocular disorders include, but are not limited to, wet age-related macular degeneration, diabetic macular edema, diabetic retinopathy, geographic atrophy, uveitis, retinitis pigmentosa and uveal melanoma. In some aspects, the ocular disorder is wet age-related macular degeneration, diabetic macular edema, diabetic retinopathy or geographic atrophy.

The terms “treating” or “treatment” refer to reducing, delaying or ameliorating an ocular disorder and/or symptoms associated therewith. In various aspects, “treating” includes slowing, delaying, or halting the progression or incidence of dysfunction in a target tissue, thereby resulting in an improvement or stabilization of a disease or disorder. The terms “prevent” or “preventing” refers to slowing or delaying the onset of a disease or dysfunction or symptom associated therewith. The composition is preferably administered as soon as possible after it has been determined that a subject is at risk for the ocular disorder (prophylactic treatment) or has begun to develop the ocular disorder (therapeutic treatment). It is appreciated that, although not precluded, “treating” or “treatment” of a disorder or condition does not require that the disorder, condition, or symptoms be completely eliminated. Similarly, “prevent” or “preventing” a disorder or condition does not require 100% protection from that disorder, condition, or symptom. Any degree of improvement in a condition, stabilization of a condition, or inhibition/slowing of the onset of a condition, is contemplated. An “effective amount” of construct or composition refers to an amount sufficient to produce a therapeutically (e.g., clinically) desirable result; the exact nature of the result will vary depending on the nature of the corneal dysfunction or ocular surface disease being treated. For example, an effective amount may be an amount sufficient to inhibit or reduce angiogenesis, reduce vascular leakage, inhibit growth factor activity, reduce lesions, and the like. An effective amount and the frequency of administration of, and the length of treatment with, a particular therapeutic agent for the treatment of an ocular disease may depend on various factors, including the eye disease, the severity of the disease, the potency of the therapeutic agent, the mode of administration, the age, body weight, general health, gender and diet of the subject, and the response of the subject to the treatment.

Exemplary methods of determining whether a subject has an ocular disorder include, but are not limited to, corneal pachymetry, corneal topography, biomicroscopy, specular microscopy, confocal microscopy and optical coherence tomography. These methods are useful in characterizing the function of, e.g., the corneal endothelium, and are appropriate for characterizing treatment or prevention of an ocular disease, disorder, or condition.

In some aspects, a composition described herein is applied to the surface of the eye. Topical administration is non-invasive or minimally invasive, increases patient compliance and avoids potential side effects of intravitreal injections, including elevated intraocular pressure, bacterial and sterile endophthalmitis, cataract formation, vitreal hemorrhage and retinal detachment. Furthermore, topical administration of a composition described herein directly to the eye can deliver a therapeutically effective amount of a payload peptide to target site(s) in the eye (e.g., in the anterior or/and posterior segments of the eye) in a much smaller dose than systemic administration of the payload peptide, and could therefore avoid or reduce potential side effects of a high dose of a payload peptide.

In some aspects, the methods described herein comprise administering a composition comprising a peptide carrier and a payload peptide, wherein the peptide carrier comprises an amino acid sequence set forth in any one of SEQ ID NOs: 1-40, and wherein the payload peptide comprises aflibercept, ranibizumab, brolucizumab, bevacizumab, faricimab, ramucirumab, fresolimumab, asunercept, recCD59, NGM621 or ANX007. In some aspects, the methods described herein comprise administering to the subject a composition comprising a peptide carrier and a payload peptide, wherein the peptide carrier comprises an amino acid sequence set forth in SEQ ID NO: 41-59, and wherein the payload peptide comprises adalimumab, infliximab, etanercept, brodalumab, ixekizumab, secukinumab, ustekinumab, guselkumab, canakinumab, toclizumab, sarilumab, RPE65, RPGR, nivolumab, atezolizumab, ipilimumab or tebentafusp. Optionally, the peptide carrier is conjugated to the payload peptide. Also optionally, the composition may comprise two or more peptide carriers and/or two or more payload peptides. For instance, the composition may comprise a peptide carrier described herein and two different payload peptides, which may act upon the same or different biological targets within the eye.

### Combination Therapies

In some aspects, the methods described herein further comprise administering one or more other therapeutic agents (in addition to the peptide carrier/payload peptide or construct described herein). Examples of additional therapeutic agents include, but are not limited to, anti-dyslipidemic agents, PPAR-alpha agonists, PPAR-delta agonists and PPAR-gamma agonists; analgesics; an agent which lowers intra-ocular pressure; anti-amyloid agents; lipofuscin inhibitors; visual/light cycle modulators and dark adaptation agents; antioxidants; antibiotics; neuroprotectants; apoptosis inhibitors; C-reactive protein (CRP) inhibitors; inhibitors of the complement system or components thereof; anti-inflammatory agents; immunosuppressants; matrix metalloproteinase (MMP) modulators (inhibitors or activators); anti-angiogenic agents; agents that preserve or improve ocular endothelium; and cell (e.g., RPE cell) replacement therapies. The composition of the disclosure may also be administered in combination with low-level light therapies, laser therapies, photodynamic therapies, radiation therapies, and surgery.

In various aspects, the method comprises administering to the subject a steroid or a non-steroid anti-inflammatory drug (NSAID). Steroids include, but are not limited to, hydrocortisone, hydrocortisone-17-butyrate, hydrocortisone-17-aceponate, hydrocortisone-17-buteprate, cortisone, tixoeortol pivaiate, prednisolone, methylprednisolone, prednisone, triamcinolone, triamcinolone acetonide, mometasone, amcinonide, budesonide, desonide, fluocinonide, haleinonide, bethamethasone, bethamethasone dipropionate, dexamethasone, fluocortolone, hydrocortisone-17-valerate, halometasone, alclometasone dipropionate, prednicarbate, clobetasone-17-butyrate, clobetasol-17-propionate, fluocortolone caproate, fluocortolone pivaiate, fluprednidene acetate, and prednicarbate.

Exemplary classes of NSAIDs that can be administered in conjunction with the composition described herein include, for instance, salicylates, propionic acid derivatives, acetic acid derivatives, enolic acid derivatives, fenamie acid derivatives, anthranilic acid derivatives and cyclooxygenase-2 (COX-2) inhibitors. Acetic acid derivatives include, e.g., aceclofenac, bromfenac, diclofenac, etodolac, indomethacin, ketorolac, nabumetone, sulindac, sulindac sulfide, sulindac sulfone and tolmetin. Anthranilic acid derivatives (fenamates) include, e.g., flufenamic acid, meclofenamic acid, mefenamic acid and tolfenamic acid. Enolic acid derivatives (oxicams) include, e.g., droxicam, isoxicam, lornoxicam, meloxicam, piroxicam and tenoxicam. Propionic acid derivatives include, e.g., fenoprofen, flurbiprofen, ibuprofen, dexibuprofen, ketoprofen, dexketoprofen, loxoprofen, naproxen and oxaprozin. Salicylates include, e.g., diflunisal, salicylic acid, acetylsalicylic acid (aspirin), choline magnesium trisalicylate, salsalate and mesalazine. COX-2 inhibitors include, e.g., apricoxib, celecoxib, etoricoxib, firocoxib, fluorocoxibs, lumiracoxib, mavacoxib, parecoxib and rofecoxib.

The combination therapy can be administered concurrently (i.e., close in time) with the composition described herein (such as during the same doctor's visit, or within about 30 or 60 minutes of each other), or prior to or subsequent to administration of the other therapeutic agent. When the composition of the disclosure is administered concurrently with another therapeutic agent, the additional therapeutic agent can be administered in the same formulation or in separate formulations as the composition described herein. In this regard, the disclosure contemplates a use of a payload peptide conjugated to an additional therapeutic agent, such as an antibody-drug conjugate. For example, the disclosure contemplates a composition comprising a peptide carrier of Formula (1) (including any one or more of SEQ ID NOs: 1-72) and a payload peptide, including any one of the payload peptides described herein (e.g., an antibody described herein), which is conjugated to drug. In various aspects, the drug is a small molecule drug, e.g., a naturally-occurring or artificially created (e.g., via chemical synthesis) molecule having a relatively low molecular weight (e.g., less than or equal to about 5 kDa) and which provides a biological effect in vivo. In various aspects, the payload peptide is conjugated to a steroid or an NSAID, such as a steroid or NSAID described herein.

Representative aspects of the disclosure are provided below:

Aspect 1. A peptide comprising no more than 50 amino acids and comprising the amino acid sequence of Formula (1): X1-X2-X3-X4-X5-X6-X7-X8-X9-X10-X11-X12-X13-X14-X15-X16-X17-F-L-I/V-P-K-G, wherein X1 is V, M, L, I, or K; X2 is K, R, or H; X3 is K, R, or H; X4 is R, K, or H; X5 is K, R, or H; X6 is I, V, or L; X7 is V, L, or I; X8 is R, K, or H; X9 is L, K, V, or R; X10 is I, L, or V; X11 is K, R, or H; X12 is F, Y, K, or L, or is absent; X13 is L, V, or I, or is absent; X14 is V, L, or I, or is absent; X15 is K, L, R, or H, or is absent; X16 is (i) M, L, V, I, R, A, K, G, C, Y, S, N, or D; is an amino acid of (i) in combination with L, K, R, or V, or (iii) is absent; and X17 is F, Y, FL, YL, FLK, YLK, FLKL, or YLKL.

Aspect 2. The peptide of Aspect 1, wherein the peptide comprises the amino acid sequence of Formula (2): X1-X2-X3-X4-X5-X6-X7-X8-X9-X10-X11-X12-X13-X14-X15-X16-X17-F-L-I-P-K-G.

Aspect 3. The peptide of Aspect 2, wherein the peptide comprises the amino acid sequence of Formula (3): L/V-K-K-R-K-I/V-V-R-L-I-K-X12-X13-X14-X15-X16-F-F-L-I-P-K-G, wherein X12 is F or Y or is absent; X13 is L or is absent; X14 is L or V or is absent; X15 is K or is absent; and X16 is L or M or is absent.

Aspect 4. The peptide of any one of Aspects 1-3, further comprising, attached at the N-terminus, L, L-L, or the amino acid sequence of Formula (4): X30-V/L-G-V/L-F-H, wherein X30 is L, I, or V.

Aspect 5. The peptide of Aspect 4, wherein the amino acid sequence attached to the N-terminus is L-V-G-V-F-H (SEQ ID NO: 102).

Aspect 6. The peptide of any one of Aspects 1-5, further comprising the amino acid sequence of one of SEQ ID NOs: 103-124 [Z1-Z23], attached at the C-terminus.

Aspect 7. The peptide of Aspect 1, wherein the peptide comprises the amino acid sequence set forth in any one of SEQ ID NOs: 1-72 comprising one, two, or three amino acid substitutions within the amino acid sequence.

Aspect 8. The peptide of Aspect 1, wherein the peptide comprises the amino acid sequence set forth in any one of SEQ ID NOs: 1-72.

Aspect 9. The peptide of Aspect 1, wherein the peptide comprises the amino acid sequence set forth in any one of SEQ ID NOs: 20, 21, 24, 30, 53, 60, 67, and 72.

Aspect 10. The peptide of Aspect 1, wherein the peptide comprises the amino acid sequence set forth in any one of SEQ ID NOs: 1-40.

Aspect 11. The peptide of Aspect 1, wherein the peptide comprises an amino acid sequence set forth in any one of SEQ ID NOs: 41-59.

Aspect 12. The peptide of Aspect 1, wherein the peptide comprises the amino acid sequence set forth in any one of SEQ ID NOs: 60-72.

Aspect 13. A construct comprising a peptide carrier of any one of Aspects 1-12 conjugated to one or more payload peptides.

Aspect 14. The construct of Aspect 13, wherein the payload peptide is an antibody or comprises an antigen-binding antibody fragment.

Aspect 15. The construct of Aspect 13, wherein the payload peptide is a recombinant protein.

Aspect 16. The construct of Aspect 13, wherein the payload peptide is aflibercept, ranibizumab, brolucizumab, bevacizumab, faricimab, ramucirumab, fresolimumab, cetuximab, panitumumab, asunercept, recCD59, NGM621 or ANX007.

Aspect 17. The construct of Aspect 16, wherein the peptide carrier comprises the amino acid sequence set forth in any one of SEQ ID NOs: 1-40.

Aspect 18. The construct of Aspect 13, wherein the payload peptide is adalimumab, infliximab, etanercept, brodalumab, ixekizumab, secukinumab, ustekinumab, guselkumab, canakinumab, toclizumab, sarilumab, RPE65, RPGR, nivolumab, atezolizumab, ipilimumab or ortebentafusp.

Aspect 19. The construct of Aspect 18, wherein the peptide carrier comprises the amino acid sequence set forth in any one of SEQ ID NOs: 41-59.

Aspect 20. The construct of any one of Aspects 13-19, wherein the payload peptide is conjugated to a drug.

Aspect 21. The construct of any one of Aspect 13-20, wherein the peptide carrier is conjugated to two or more payload peptides.

Aspect 22. A composition comprising the construct of any one of Aspects 13-21 and a pharmaceutically acceptable carrier, diluent or excipient.

Aspect 23. A composition comprising a peptide carrier of any one of Aspects 1-12, a payload peptide, and a pharmaceutically acceptable carrier, diluent or excipient.

Aspect 24. The composition of Aspect 23, wherein the payload peptide is an antibody or comprises an antigen-binding antibody fragment.

Aspect 25. The composition of Aspect 23, wherein the payload peptide is a recombinant protein.

Aspect 26. The composition of Aspect 23, wherein the payload peptide is aflibercept, ranibizumab, brolucizumab, bevacizumab, faricimab, ramucirumab, fresolimumab, cetuximab, panitumumab, asunercept, recCD59, NGM621 or ANX007.

Aspect 27. The composition of Aspect 26, wherein the peptide carrier comprises the amino acid sequence set forth in any one of SEQ ID NOs: 1-40.

Aspect 28. The composition of Aspect 23, wherein the payload peptide is adalimumab, infliximab, etanercept, brodalumab, ixekizumab, secukinumab, ustekinumab, guselkumab, canakinumab, toclizumab, sarilumab, RPE65, RPGR, nivolumab, atezolizumab, ipilimumab or ortebentafusp.

Aspect 29. The composition of Aspect 28, wherein the peptide carrier comprises the amino acid sequence set forth in any one of SEQ ID NOs: 41-59.

Aspect 30. The composition of any one of Aspects 23-29, wherein the payload peptide is conjugated to a drug.

Aspect 31. The composition of any one of Aspects 22-30, which is formulated as an eye drop.

Aspect 32. A method of delivering a payload peptide to an eye of a subject, the method comprising administering the composition of any one of Aspects 22-31 to the eye of the subject.

Aspect 33. A method of treating an ocular disorder in a subject in need thereof, the method comprising administering the composition of any one of Aspects 22-31 to an eye of the subject.

Aspect 34. The method of Aspect 33, wherein the ocular disorder is wet age-related macular degeneration, diabetic macular edema, diabetic retinopathy, retinal vein occlusion, geographic atrophy, uveitis, endophthalmitis, retinal detachment, optic neuropathy, optic neuritis, neuromyelitis optica, retinitis pigmentosa or uveal melanoma.

Aspect 35. The method of Aspect 33, wherein the ocular disorder is wet age-related macular degeneration, diabetic macular edema, diabetic retinopathy or geographic atrophy.

Aspect 36. The method of Aspect 35, wherein the payload peptide is aflibercept, ranibizumab, brolucizumab, bevacizumab, faricimab, ramucirumab, fresolimumab, cetuximab, panitumumab, asunercept, recCD59, NGM621 or ANX007, and the peptide carrier comprises the amino acid sequence set forth in any one of SEQ ID NOs: 1-40.

Aspect 37. The method of Aspect 33, wherein the ocular disorder is uveitis, retinitis pigmentosa or uveal melanoma.

Aspect 38. The method of Aspect 37, wherein the payload peptide is adalimumab, infliximab, etanercept, brodalumab, ixekizumab, secukinumab, ustekinumab, guselkumab, canakinumab, toclizumab, sarilumab, RPE65, RPGR, nivolumab, atezolizumab, ipilimumab or ortebentafusp and the peptide carrier comprises the amino acid sequence set forth in any one of SEQ ID NOs: 41-59.

Aspect 39. The method of any one of Aspects 32-38, wherein the composition is topically administered to the subject.

Aspect 40. The method of any one of Aspects 33-39, further comprising administering a non-steroid anti-inflammatory drug (NSAID) to the subject.

Aspect 41. The method of Aspect 40, wherein the NSAID is a salicylate, a propionic acid derivative, an acetic acid derivative, an enolic acid derivative, a fenamie acid derivative or a cyclooxygenase-2 (COX-2) inhibitor.

Aspect 42. The method of Aspect 40 or Aspect 41, wherein the NSAID is conjugated to the payload peptide.

Aspect 43. The method of any one of Aspects 33-39, further comprising administering a steroid to the subject.

Aspect 44. The method of Aspect 43, wherein the steroid is hydrocortisone, hydrocortisone-17-butyrate, hydrocortisone-17-aceponate, hydrocortisone-17-buteprate, cortisone, tixoeortol pivaiate, prednisolone, methylprednisolone, prednisone, triamcinolone, triamcinolone acetonide, mometasone, amcinonide, budesonide, desonide, fluocinonide, haleinonide, bethamethasone, bethamethasone dipropionate, dexamethasone, fluocortolone, hydrocortisone-17-valerate, halometasone, alclometasone dipropionate, prednicarbate, clobetasone-17-butyrate, clobetasol-17-propionate, fluocortolone caproate, fluocortolone pivaiate, fluprednidene acetate or prednicarbate.

Aspect 45. The method of Aspect 43 or Aspect 44, wherein the steroid is conjugated to the payload peptide.

The technology described herein is further illustrated by the following examples which in no way should be construed as being further limiting.

### EXAMPLES

### Example 1—Intraocular Penetration Activity of Various Constructs

Antibodies: Aflibercept biosimilar PMC-902, anti-Tie-2 antibody PMC-403, anti-VEGFR2 antibody olinvacimab, and angiopoietin-2 antibody PMC-401s were obtained from Pharmabcine, South Korea. Other antibodies were commercially available: Eylea® (aflibercept, Regeneron, NJ), Beovu® (brolucizumab, Novartis), adalimumab biosimilar (BioXcell, NH), ranibizumab, bevacizumab, cetuximab, pembrolizumab, nivolumab, ipilimumab, brodalumab and secukinumab biosimilar antibodies (IchorBio, UK). Recombinant human VEGF165 was purchased from Genscript. Recombinant proteins including adiponectin, angiostatin, endostatin, GM-CSF and IL-2 were purchased from Acro Biosystems (Newark, DE). IRDye 680RD and IRDye 8000W were purchased from LiCor (Lincoln, NE).

Animals: Eight-week-old female C57BL/6 (Charles River Laboratories) and C57BL/6J (Jackson Laboratory Bar Harbor, ME) female mice were acclimated for at least 3 days and fed ad-lib on a chow diet with a 12 hr light/dark cycle. All studies and procedures were performed in accordance with the Mispro IACUC approved protocol and standard ethical guidelines. Young adult Yorkshire pigs (12-16 weeks old, male) were treated and cared for in accordance with NIH Guide for the care and use of laboratory animals (8^{th} edition, Washington DC, 2011), and all protocols were approved by the University of South Carolina Institutional Animal Care and Use Committee (IACUC).

Peptide synthesis: The modified and non-modified peptide carriers were synthesized using either Fmoc or Boc solid phase peptide synthesis (SPPS) methods by Genscript (Piscataway, NJ). A representative synthesis method is described. The peptide synthesis was based on the resin as a solid support and linkers are modified on the resin to provide the reactive group for the first amino acid through the amide bond between the amide group and carboxyl group from another in the condition of pre-activated species or in situ activation. To avoid the undesired reactions, protecting groups are involved to temporary mask reactive groups on both N a-position and side chain of the amino acid. For Fmoc synthesis, the first Fmoc amino acid is attached to an insoluble support resin via an acid labile linker. Deprotection of Fmoc is accomplished by treatment of the amino acid with a base, usually 20-50% piperidine and the progress of each deprotection reaction can be followed by real time spectrophotometric monitoring the release of the cleaved Fmoc-group at 300-320 nm. The second Fmoc amino acid is coupled utilizing a pre-activated species or in situ activation. After the desired peptide is synthesized, the resin bound peptide is deprotected and detached from the solid support via TFA cleavage. The cleavage was done at RT for two hours in the cocktail of Reagent K (5 mL of TFA/H2O/thioansole/phenol/EDT to 82.5:5:5:5:2.5). For Boc synthesis, N alpha-position protecting group of the first Boc amino acid is attached to an insoluble support resin via a HF cleavable linker. Deprotection of Boc amino acid is accomplished by treatment of the amino acid with TFA. The second Boc amino acid is coupled utilizing a pre-activated species or in situ activation. After the desired peptide is synthesized, the resin bound peptide is deprotected and detached from the solid support via HF cleavage. The resin was filtered, and the peptide was precipitated by the addition of cold ether (50 mL). The precipitate was collected by centrifugation and then was washed with cold ether (3×50 mL). The crude peptide was dissolved in 20 mM alkaline glycine buffer (500 mL) followed with the addition of solid cysteine-HCl (0.75 mmol). The pH of the solution was adjusted to 10.5 with 1 M NaOH. Sonication was used to accelerate the dissolution of the crude peptide. The resulting solution was stirred vigorously at 4° C. for 2 d. The pH was lowered to 7.0 by the addition of 1 M HCl, and the solution was subjected to purification by preparative reverse-phase HPLC column [Luna 10-μm C8 100 Å, LC column (Phenomenex Inc.), 250×21.2 mm, 10-50% aqueous acetonitrile (0.1% TFA) over 90 min, at a flow rate of 15 mL/min]. Ion exchange HPLC is also used when reverse-phase HPLC is inapplicable, such as in the purification of very long peptides or large quantities of peptide. The quality was confirmed by mass spectrometry(MS) and analytical high performance liquid chromatography (HPLC) analysis. Solubility was tested in water, PBS and DMSO. Synthesized peptides were further conjugated with fluorescent dyes including Cy5, Cy7, AF488 and AF647 for image analyses.

Eye drop formulation: For peptide carrier screening, Fab and scFv Abs (ranibizumab, brolucizumab, and PMC-401s) were formulated 3-5 ug into 3 uL volume, and full IgG Abs (adalimumab, bevacizumab, cetuximab, pembrolizumab, nivolumab, ipilimumab, ramucirumab, brodalumab and secukinumab biosimilar antibodies) or Fc fusion protein (aflibercept) were formulated 5-15 ug IR680 or IR800 (LI-COR, Lincoln, NE) labeled antibody with a 20× molar concentration of peptide carrier in Buffers 1-8 listed below. For aflibercept biosimilar PMC-902 eye drop efficacy test, each 3 ul eye drop was formulated with 5-20 ug unlabeled antibody with 12×, 20× or 35× molar concentration of peptide carrier in Buffer 1, 2, 3, 6 and 8 listed below. Brolicizumab was formulated with 5-10 ug antibody with 12×, 20× or 40× molar concentration of peptide carrier in Buffer 1, 2, 3 and 7 listed below. PMC-403 eye drops for efficacy studies were formulated with 10 ug unlabeled antibody with 20× molar concentration of peptide carrier in Buffers 1, 2, 4, and 5 listed below with 1% Captisol® (Ligand Pharmaceutical, San Diego, CA).

TABLE 5
Buffers

| | |
|---|---|
| Buffer 1 | 1.06 mM potassium phosphate dibasic, 154 mM sodium chloride, 2.97 mM |
| | sodium phosphate dibasic, pH 7.4 |
| Buffer 2 | 10 mM sodium phosphate, 40 mM sodium chloride, 0.03% polysorbate 20, |
| | and 5% sucrose, pH 6.2 |
| Buffer 3 | 0.025% Brimonidine tartrate, benzalkonium chloride, boric acid, calcium |
| | chloride dihydrate, glycerin, potassium chloride, benzalkonium chloride, |
| | boric acid, calcium chloride dihydrate, glycerin, potassium chloride, sodium |
| | borate decahydrate, sodium chloride |
| Buffer 4 | 20 mM L-histidine, 45 g/L sucrose, 0.06% PS80, 70 mM Arg-HCl, pH 6.0 |
| Buffer 5 | 10 mM sodium acetate, 9% sucrose, pH 5 |
| Buffer 6 | 0.9% sodium chloride |
| Buffer 7 | 0.02% polysorbate 80, 10 mM sodium citrate, 5.8% sucrose, pH 7.2 |
| Buffer 8 | 0.1% Dextran70, 0.2% Glycerin, 0.3% Hypromellose, potassium chloride, |
| | purified water, sodium borate, sodium chloride |

Whole eyeball imaging for ocular distribution in mouse: Eye drop formulations were applied to mouse eyes and enucleated at desired time points (15-60 min) after euthanizing by CO₂. Enucleated eyeballs were placed in 4% paraformaldehyde (PFA) and fixed at room temperature for up to two hours. PFA was then replaced with PBS. Using a tissue matrix, razor blades and tweezers, one eyeball at a time was placed into the tissue matrix with the cornea side down. Once aligned into the center of the matrix, a clean razor blade was used to cut the eyeball in half. With tweezers, each cut half of the eye was gently dipped in Tissue Tek optimal cutting temperature compound and placed with the interior side down on a clear plastic sheet for imaging. Sliced eye images were acquired in a LiCor Odyssey with the following settings: Omm height, 21 um resolution, medium quality, 800 channel.

Collection and quantitation of aqueous humor, vitreous and retina from mouse eyes: Mice were euthanized by CO₂. Eyeballs were enucleated and rinsed 3× with PBS. Eyes were gently dried and placed on a petri dish. Using slanted tweezers, the eyeball was held in place on its side. Using a 31 g 0.3 cc insulin syringe, the beveled edge was angled at 45 degrees and entered the anterior chamber from the limbus. The plunger was pulled back to draw up the aqueous into the barrel of the syringe. The needle was removed from the eyeball and the aqueous humor was deposited into the center of a well in a black 96 well plate (Corning). With straight edged spring scissors, the tip of one of scissor blades was inserted into the hole left by the syringe and cuts were made following the limbus all around the eye to create two eye cups. Using straight tweezers, the vitreous/lens was pulled out of one eyecup and the retina was gently scraped off the other eyecup. The vitreous and retina were placed into the center of a well in a black 96w plate. The plate was imaged in a LiCor Odyssey with the following settings: 4 mm height, 169 um resolution, low quality, 800 channel. Imaged was analyzed using Image Studio Lite Ver 5.2 software.

Ocular penetration screen of antibody/carrier peptide formulation in mouse eyes: IR800-aflibercept biosimilar (PMC-902), and IR800-brolucizumab were formulated with carrier in PBS. C57BL/6 (Charles River Laboratories, Wilmington, MA) and C57BL/6J (Jackson Laboratory Bar Harbor, ME) female mice were anesthetized with isoflurane. A 3 ul eye drop containing 5 ug IR800 or IR680 (LI-COR) labeled antibody formulated with 20× molar concentration of carrier was applied per eye. Animals were placed in a heated recovery chamber and re-housed. After 15 minutes, animals were euthanized and enucleated. After collection of the aqueous humor with a 31G insulin syringe, eyes were dissected, and retina, vitreous, and leftover eye tissues placed in separate wells of a black 96-well plate. Plates were scanned with an Odyssey CLx (LI-COR).

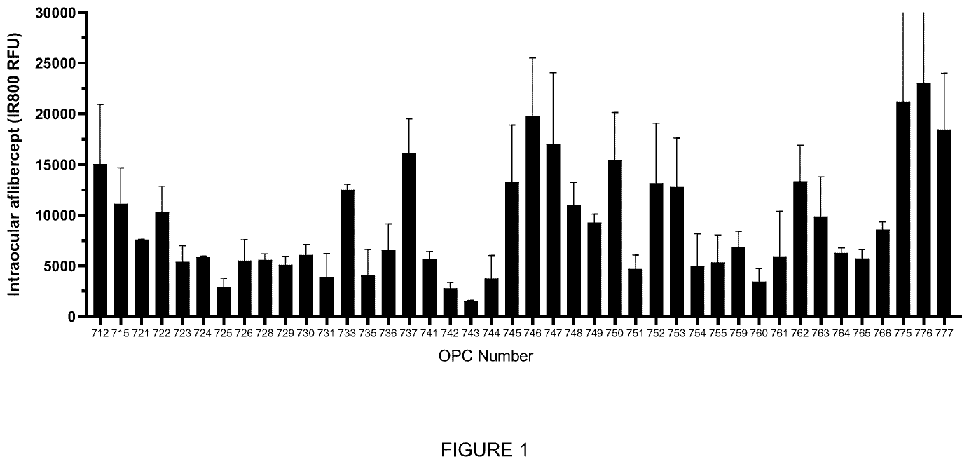

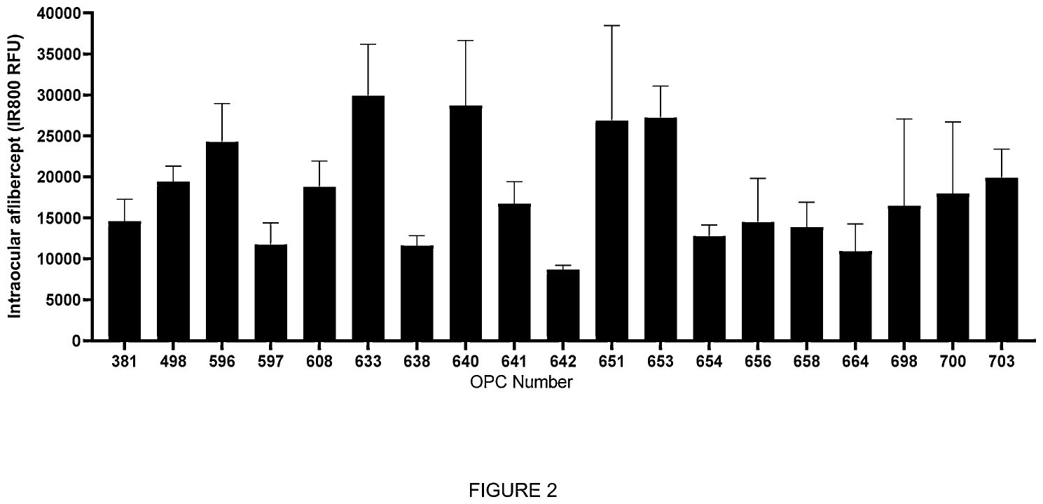

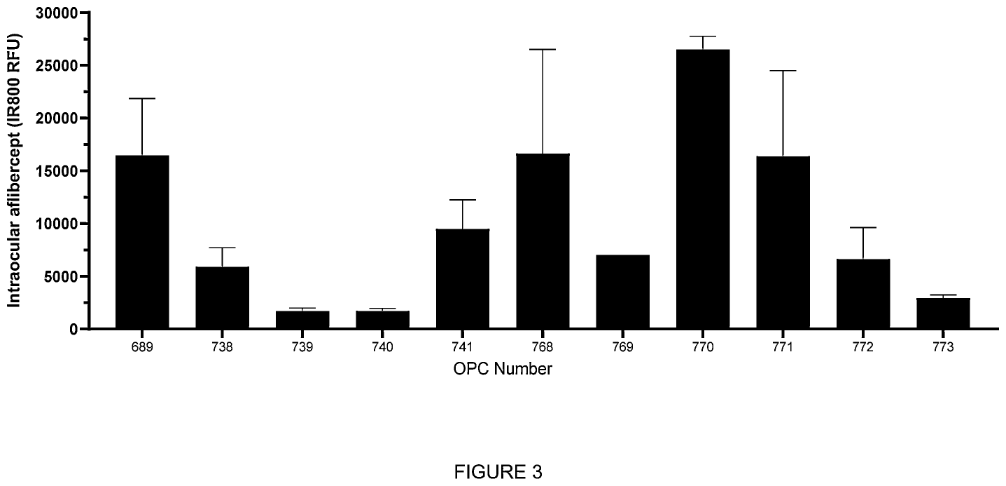

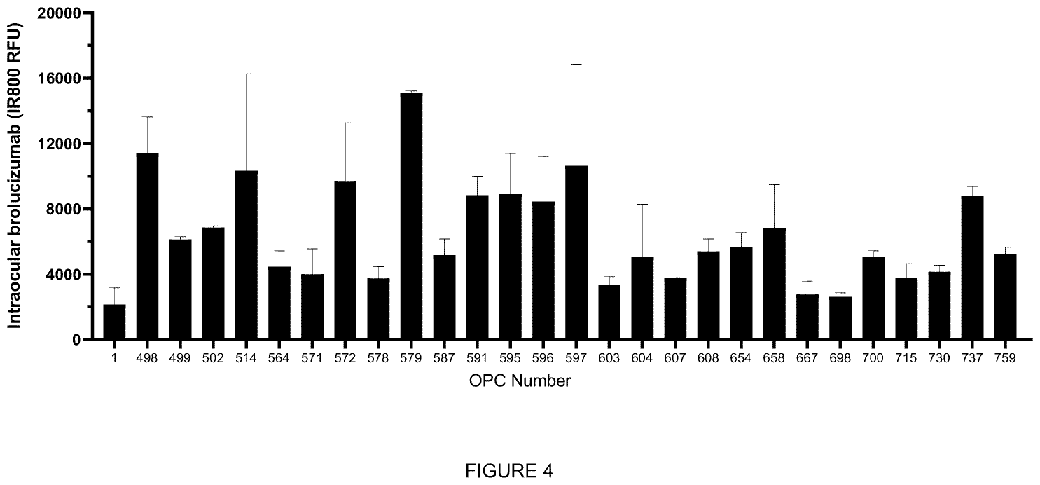

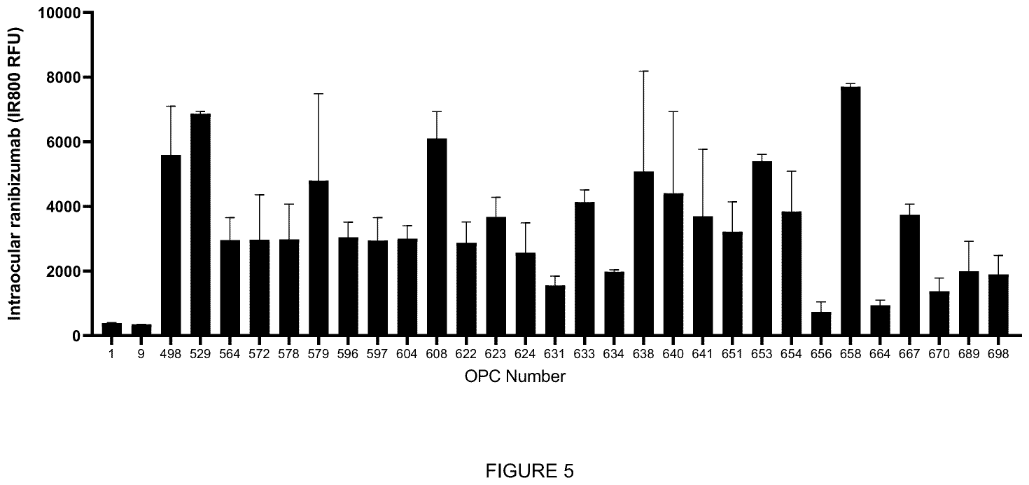

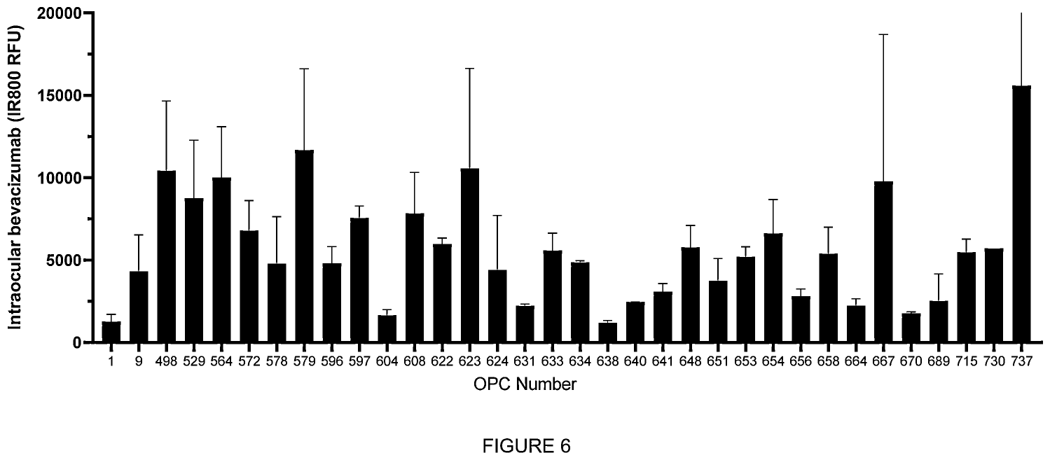

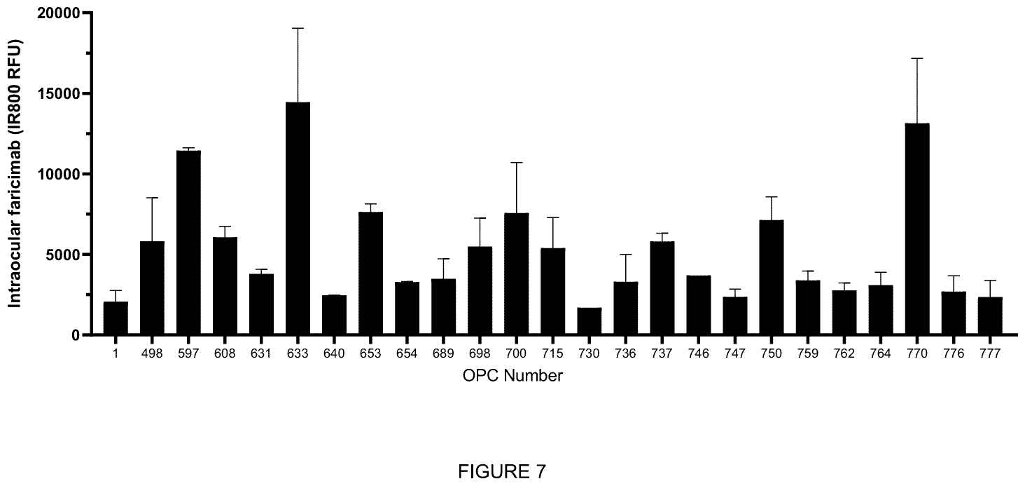

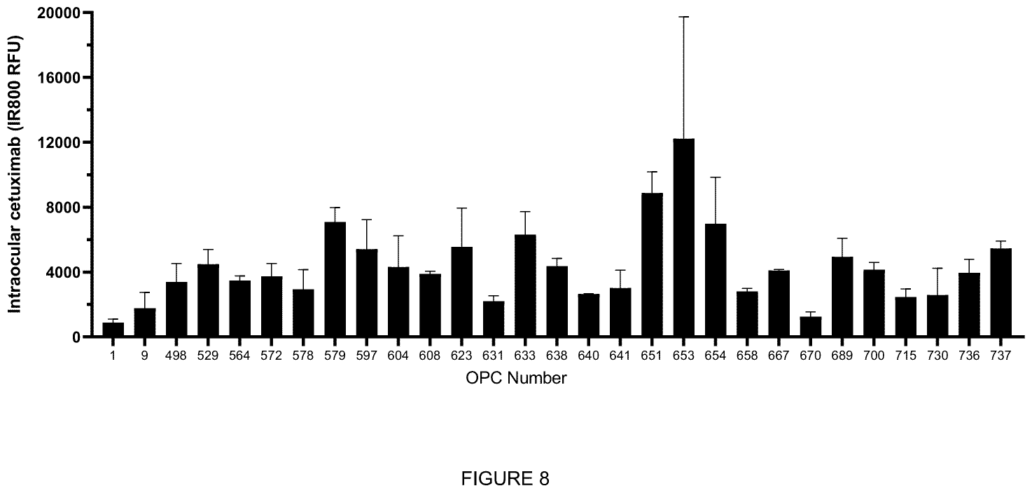

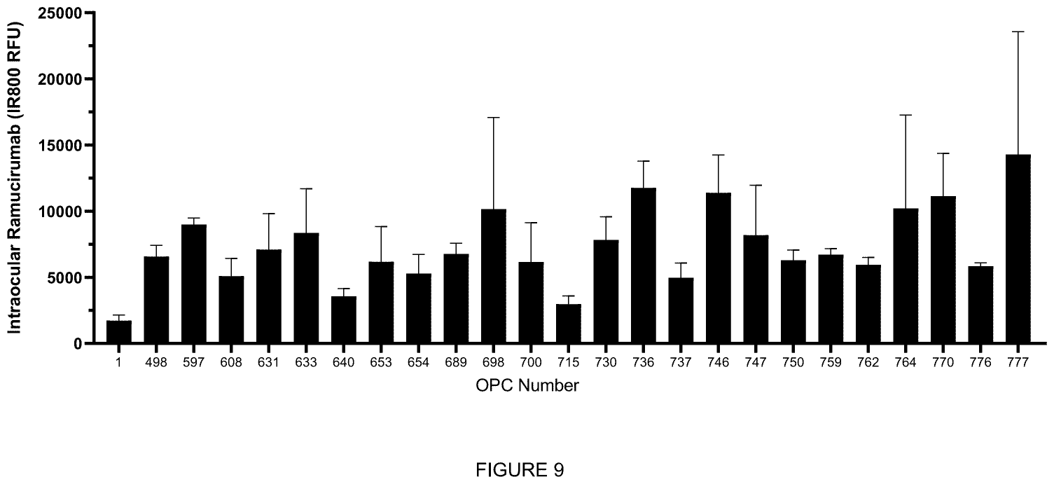

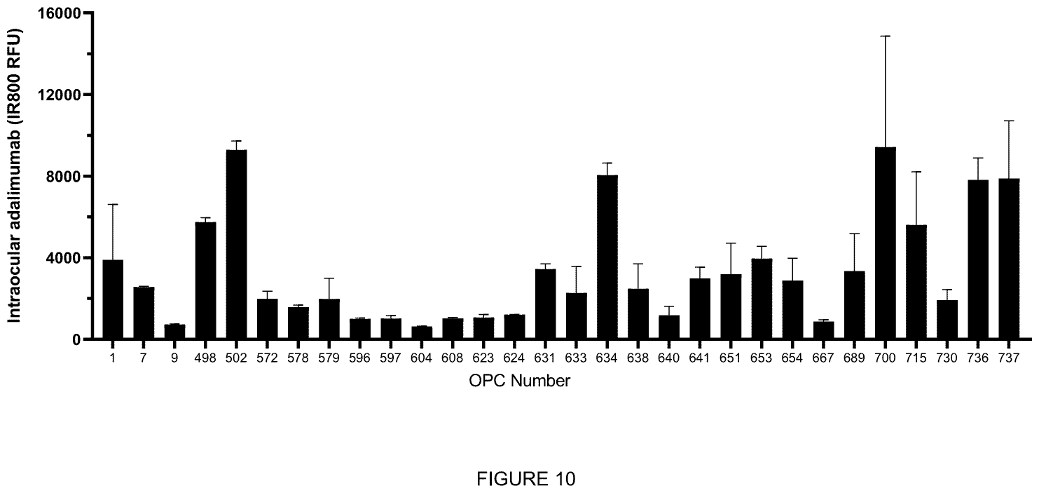

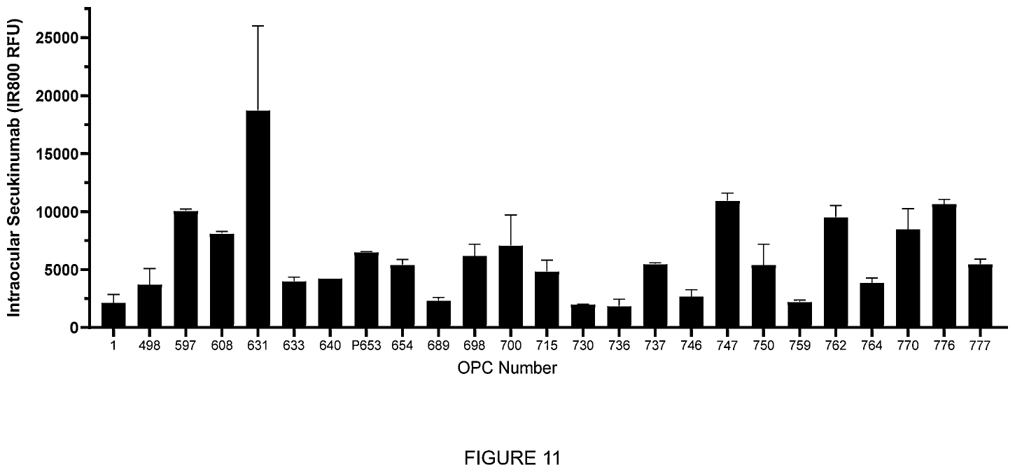

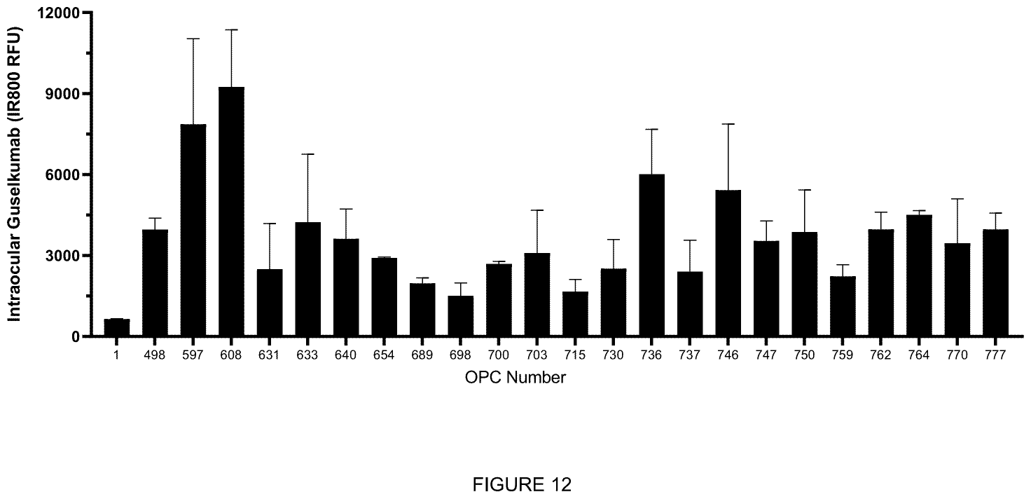

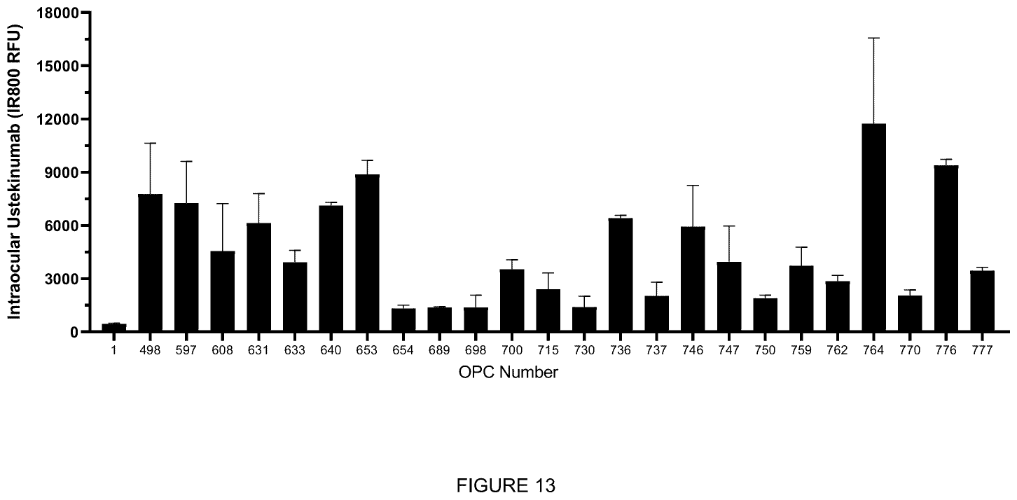

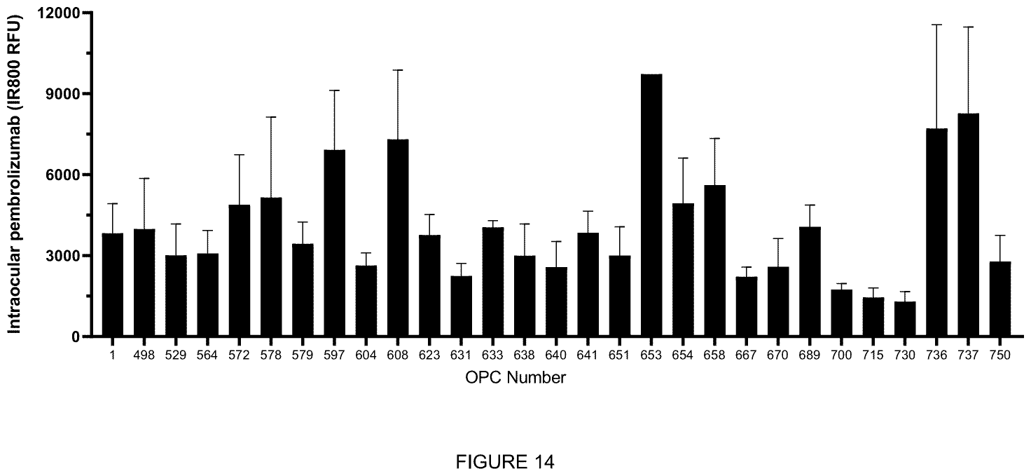

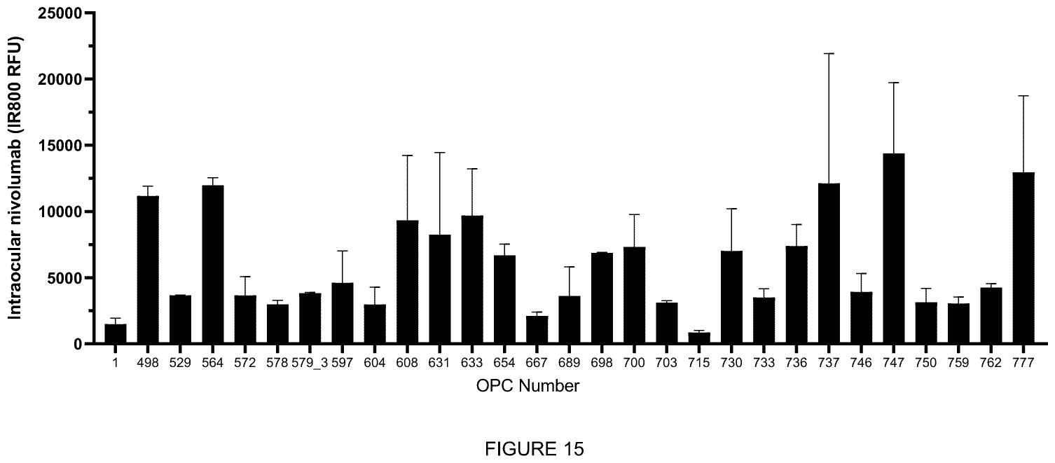

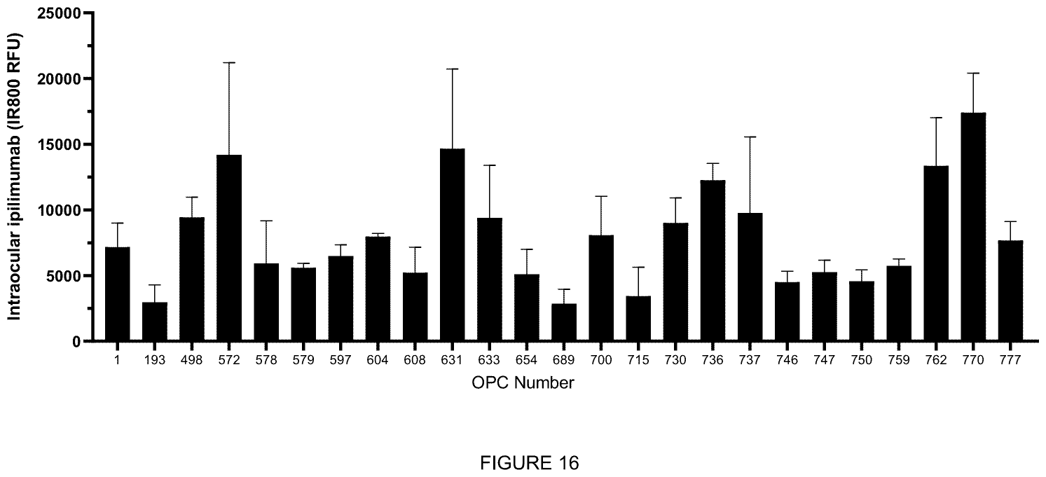

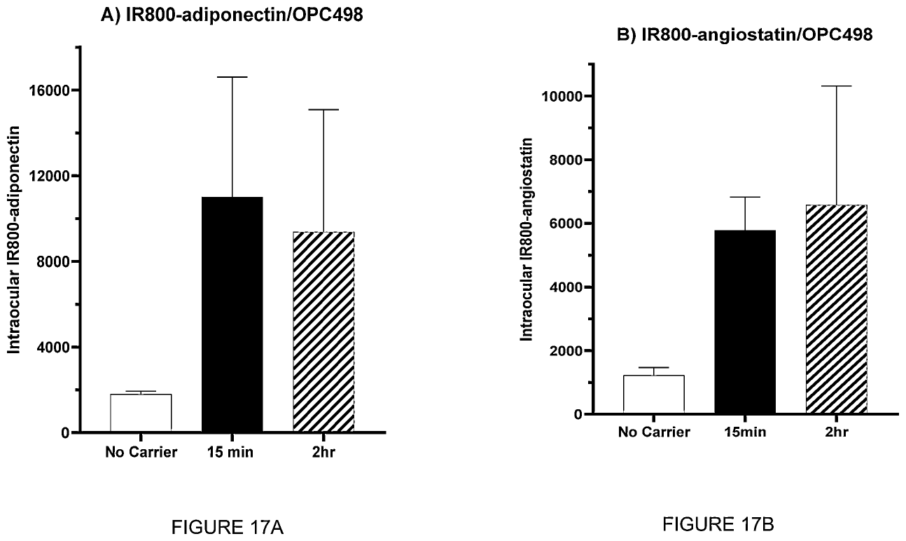

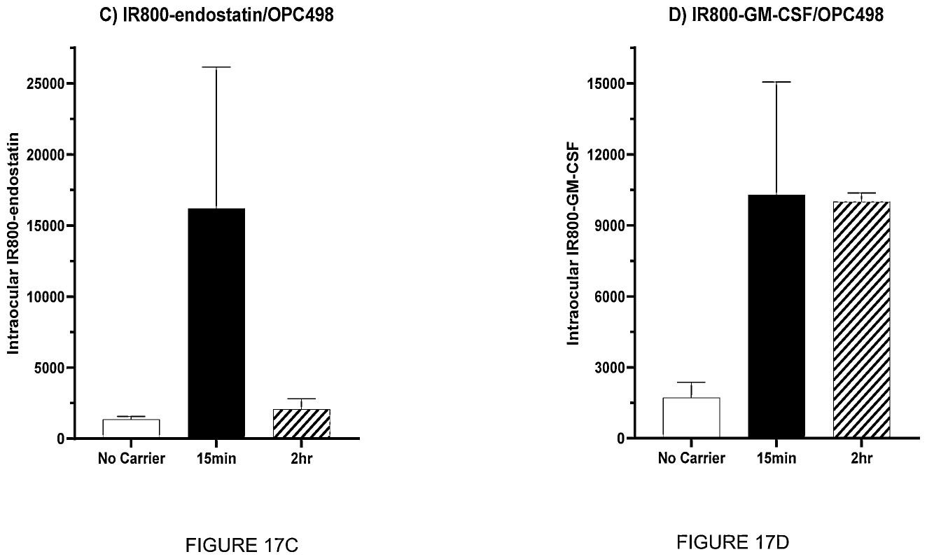

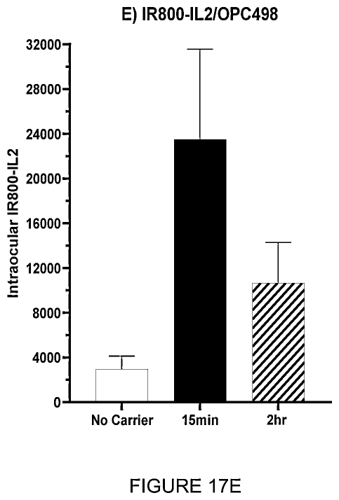

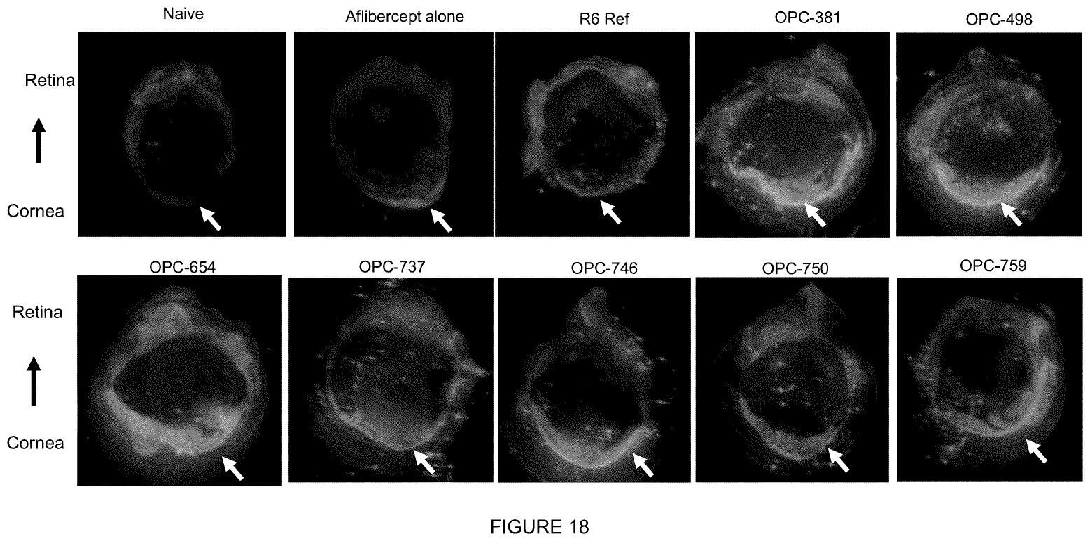

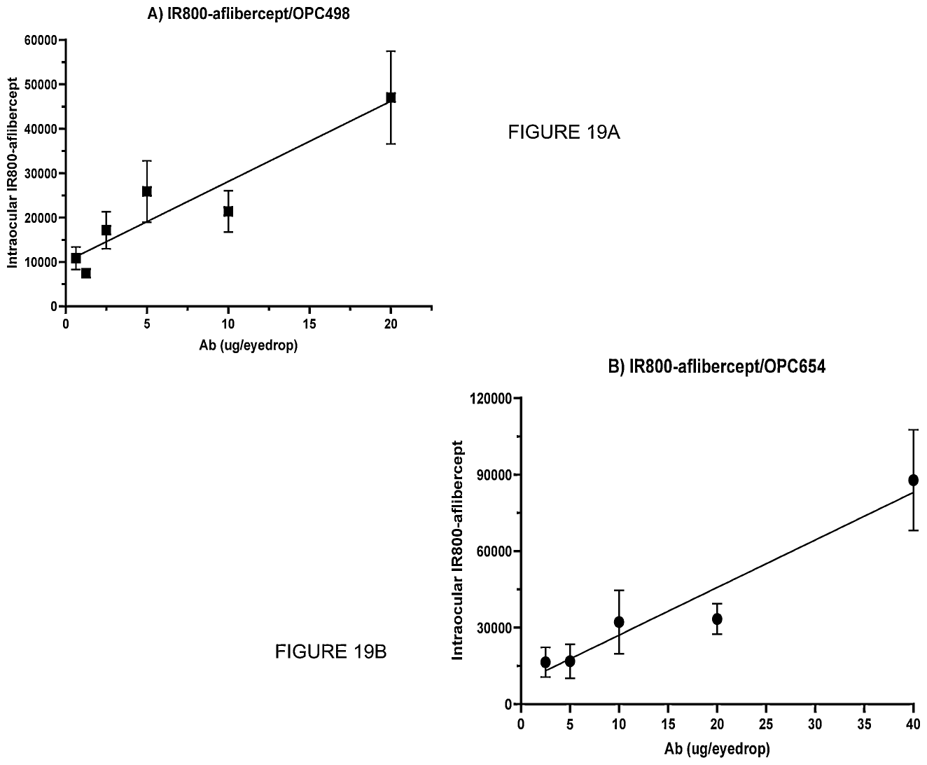

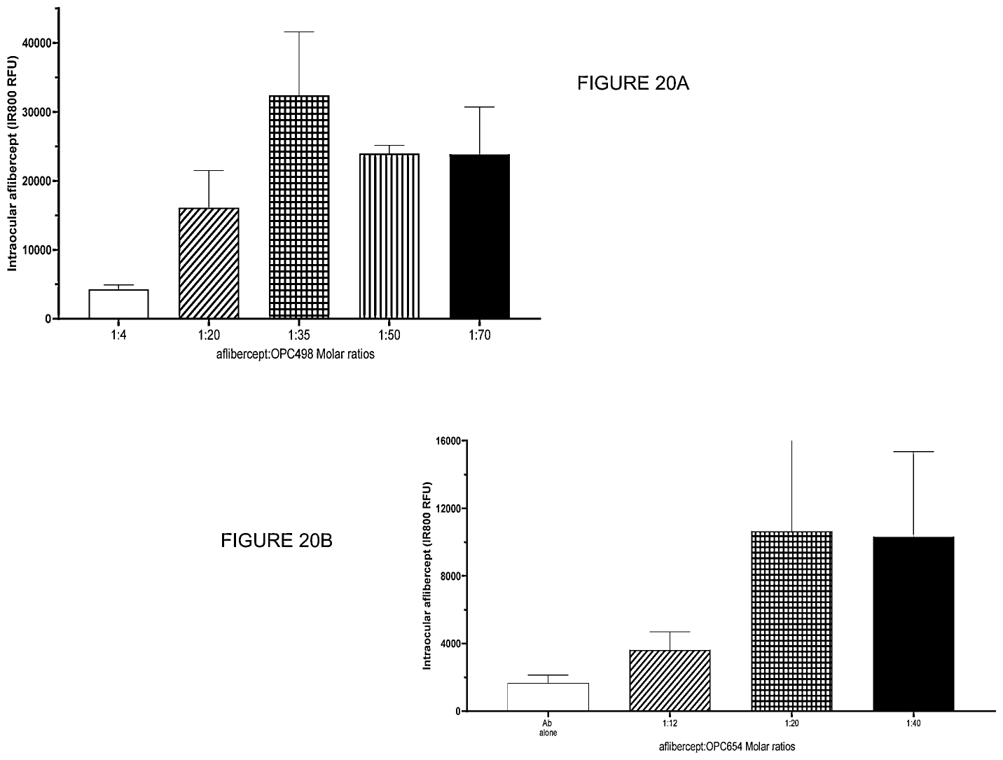

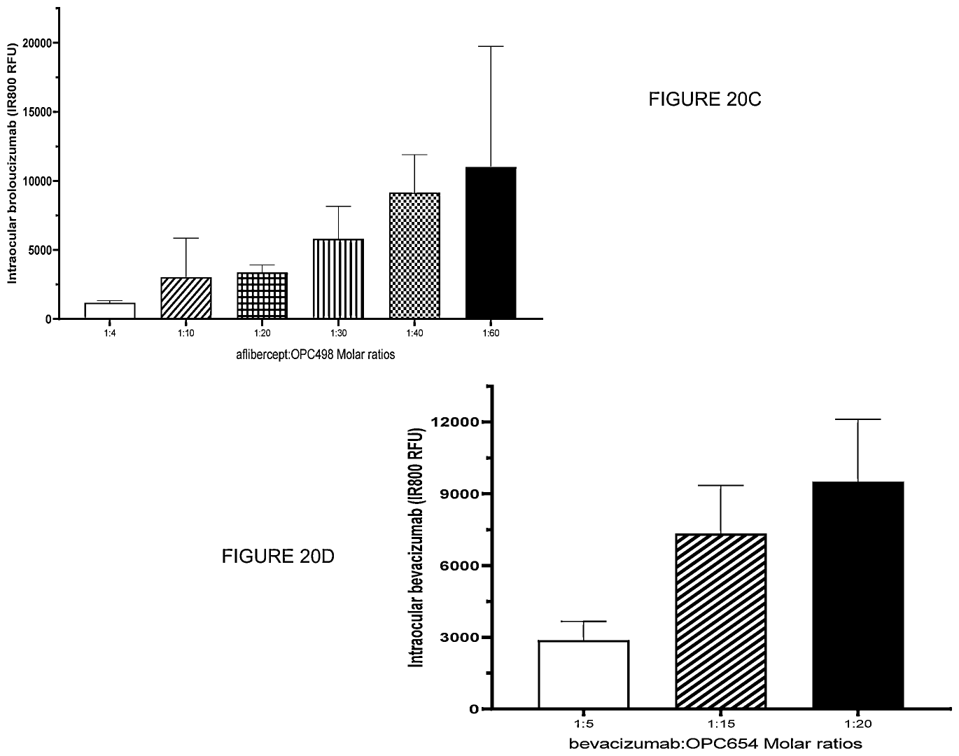

The ability of peptide carriers of the disclosure to mediate penetration of a number of different antibodies into the eye is illustrated in FIGS. 1-16. The methods also were performed with different recombinant proteins, including adiponectin, angiostatin, endostatin, GM-CSF, and IL-2. The results are illustrated in FIGS. 17A-E.

Table 6 below provides a further summary of intraocular penetration of IR800-antibodies (anti-TGF-beta, anti-FasL, anti-Ang2 scFv PMC-401s, Tie 2 agonist antibody PMC-403, and anti-VEGFR2 Olinvacimab) administered with PC (peptide carrier, also referred to herein as OPC) in an eye drop formulation in mouse eyes. Vitreous and retina samples were collected 15 min after eye drop application of IR800 labeled Ab-PC formulation (1:20 ratios) in 1× PBS (10 ug in 3 uL per eye). Intraocular IR800-Ab signal±SEM, N=2-6 eyes.

This example demonstrates that peptide carriers of the disclosure increase intraocular penetration of a variety of different payload peptides, including antibodies, antibody fragments, cytokines (e.g., IL-2 and GM-CSF), hormones (e.g., adiponectin), as well as other recombinant proteins (e.g., angiostatin and endostatin).

TABLE 6

| | | | PMC-401s | | |
|---|---|---|---|---|---|
| | FasAb | TGFb Ab | (Target: | PMC-403 | Olinvacimab |
| Peptide | (Target: | (Target: | Angiopoietin- | (Target: | (Target: |
| ID | FasL) | TGFβ) | 2) | Tie-2) | VEGFR2) |
| 1 | + | + | + | + | + |
| 9 | + | | | + | |
| 381 | | | +++ | ++ | |
| 498 | ++ | +++ | +++ | ++ | ++ |
| 502 | | | +++ | | |
| 572 | ++ | +++ | | ++ | |
| 578 | ++ | +++ | | +++ | |
| 579 | +++ | +++ | | ++ | |
| 597 | | +++ | | ++ | |
| 604 | | | | ++ | |
| 608 | +++ | +++ | ++ | ++ | ++ |
| 622 | ++ | +++ | | | +++ |
| 623 | | | | ++ | |
| 624 | | | | +++ | |
| 631 | | | | ++ | |
| 633 | +++ | +++ | +++ | ++ | + |
| 634 | +++ | +++ | | | |
| 638 | ++ | ++ | | | |
| 640 | +++ | +++ | | ++ | + |
| 641 | +++ | +++ | | ++ | |
| 648 | | ++ | | ++ | |
| 651 | +++ | +++ | | | + |
| 653 | +++ | +++ | | +++ | ++ |
| 654 | +++ | ++ | ++ | +++ | ++ |
| 658 | ++ | ++ | | | ++ |
| 667 | +++ | ++ | | | + |
| 670 | + | + | | | |
| 689 | ++ | ++ | ++ | +++ | + |
| 698 | +++ | | | | ++ |
| 736 | | ++ | ++ | +++ | + |
| 737 | +++ | | ++ | +++ | + |
| 746 | | + | ++ | ++ | + |
| 747 | | ++ | +++ | +++ | + |
| 750 | | | ++ | +++ | + |
| 759 | + | ++ | +++ | +++ | + |
| 762 | | + | ++ | ++ | + |
| 777 | + | + | ++ | +++ | + |
| 685 | | | | | ++ |
| 798 | | ++ | | | +++ |
| 808 | + | | | + | +++ |
| 825 | | | | + | + |

### Example 2—Detection of Intraocularly Delivered Antibody by ELISA

Aflibercept biosimilar (PMC-902) was formulated with peptide carriers in PBS and incubated overnight at 4° C. One eye drop per eye was applied to C57Bl/6 animals under isoflurane anesthesia. At specified time points after eye drop treatment, animals were euthanized, enucleated, and dissected to harvest vitreous and retina. Vitreous and retina were homogenized in RIPA buffer (Millipore) supplemented with Protease Inhibitor Cocktail (Sigma). Homogenates were clarified by centrifugation. ELISA plates were coated with goat anti-human IgG Fc (Invitrogen) and blocked with 5% BSA (Prometheus) in PBS supplemented with 0.05% Tween-20 (PBST). Samples and dilutions of the antibody standard were loaded onto the ELISA plate and incubated on a platform shaker. After extensive washes with PBST, the detection antibody, goat-anti human IgG Fc HRP-conjugated (Thermo), was added and incubated at room temperature. Plates were washed extensively before adding 1-Step Ultra TMB-ELISA solution (Thermo). After 15-30 minutes incubation the ELISA STOP solution was added (Thermo) and plates were scanned using a Thermo AccuSkan FC ELISA plate reader. A similar method was performed using an anti-Tie 2 antibody (PMC-403) (10 ug, 1:20 ratios) with peptide carriers 572, 578 and 633 in PBS and His/Arg buffer conditions.

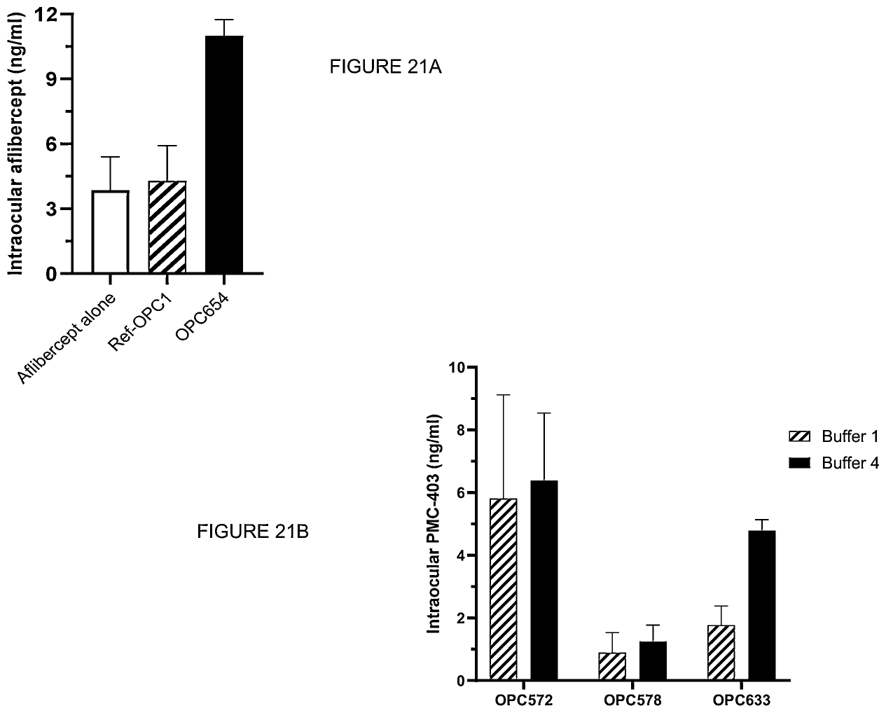

Results are shown in FIGS. 21A and 21B. Peptide carrier 654 significantly enhanced aflibercept intraocular levels. PMC-403 was detected intraocularly when combined with peptide carriers 572, 578, and 633.

### Example 3—VEGF Binding Activity of Eye Drop Formulated Anti-VEGF Antibody

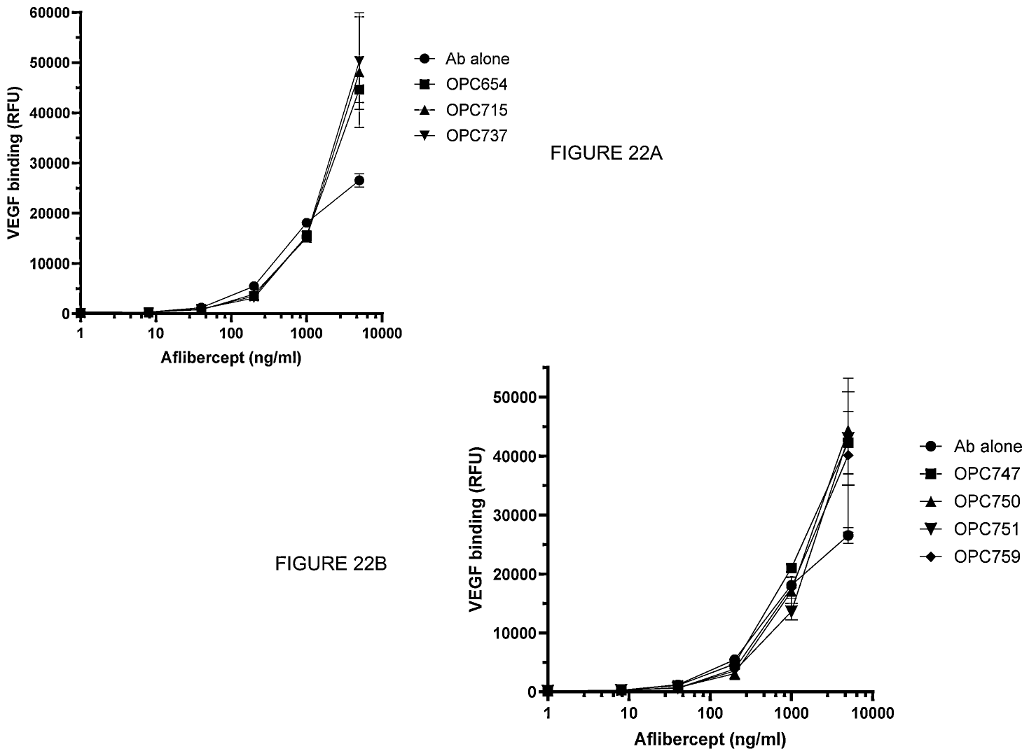

IR800 labeled antibody-carrier eye drops were formulated in PBS at concentrations similar to in vivo studies. High binding ELISA plates were coated with recombinant antigen diluted in PBS for 2 hours at room temperature or overnight at 4° C. Antigen-coated plates were washed multiple times with PBS supplemented with 0.05% Tween-20 (PBST) and blocked for 1 hour at room temperature with 5% BSA (Prometheus, Genesee Scientific) in PBST. Dilutions of antibody-peptide carrier eye drops were added to blocked ELISA plates and incubated at room temperature for 2 hours. After multiple washes, plates were scanned on the LI-COR Odyssey CLx. Human recombinant VEGF (Genscript) antigen was used for aflibercept biosimilar (PMC-902). Results are shown in FIGS. 22A-22B that eyedrop formulations with OPC654, 715, 737, 747, 750, 751, and 759 do not block aflibercept binding activity and that aflibercept/OPCs are functional.

### Example 4—Vitreoretinal VEGF Clearance by Aflibercept/Peptide Carrier Eye Drops

As a surrogate for VEGF clearance, recombinant mouse VEGF (R&D Systems, Minneapolis, MN) was diluted in PBS and IVT injected into the mouse eye with a 36G needle affixed to a NanoFil syringe (World Precision Instruments, Sarasota, FL). Aflibercept/peptide carrier eye drops formulated in PBS were applied after IVT injection. To detect clearance by aflibercept/peptide carrier eye drops, eyeballs were enucleated, vitreous and retina were harvested and homogenized in RIPA buffer (Millipore/Sigma) supplemented with protease inhibitor cocktail (Sigma). A VEGF ELISA was used to detect intraocular VEGF levels. Goat anti-mouse VEGF antibody (Invitrogen/Thermo Fisher) was diluted in PBS and applied to a high binding ELISA plate. After overnight incubation, the plate was washed with PBST and blocked with 5% BSA PBST. Samples and a VEGF standard were diluted in RIPA buffer, applied to the ELISA plate, and incubated at room temperature for two hours. The ELISA plate was washed multiple times with PBST and incubated with biotinylated goat anti-mouse VEGF (R&D Systems) for 1 hour at room temperature. The ELISA plate was washed multiple times with PBST and incubated with Streptavidin-HRP (R&D Systems) for 30 minutes at room temperature. The ELISA plate was washed multiple times before adding 1-Step Ultra TMB Substrate (Thermo Fisher) and incubated 15 minutes at room temperature. Stop Solution for TMB Substrates was added (Thermo Fisher) and the plate was scanned using an Accuskan Fc microplate reader (Thermo Fisher).

To detect VEGF clearance using the LI-COR Odyssey CLx, IR800-labeled mouse recombinant VEGF was diluted in PBS and IVT injected into the mouse eye. Aflibercept/peptide carrier eye drops formulated in PBS were applied after intravitreal (IVT) injection. After a short incubation, eyeballs were enucleated and dissected. Aqueous humor, vitreous, retina, and remaining eye tissue were placed in black 96-well microplates and scanned on the Odyssey CLx (LI-COR).

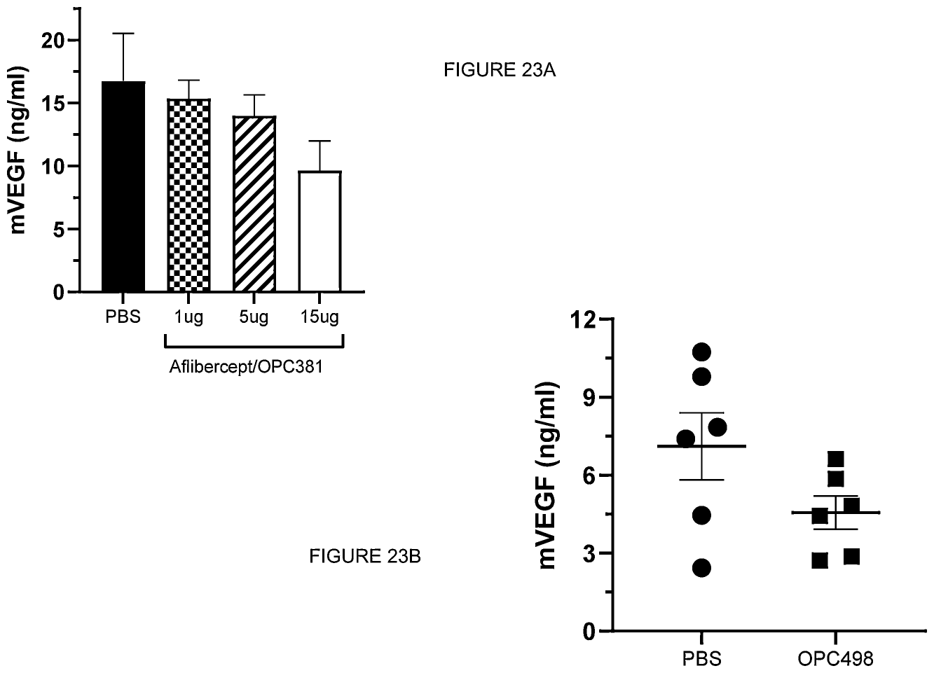

Results are shown in FIGS. 23A and 23B. Administration of a composition comprising aflibercept and peptide carrier 381 or peptide carrier 498 mediated a reduction in mVEGF levels when applied topically to the eye surface. The data illustrate that the composition of the disclosure comprising the peptide carriers of the disclosure and a payload peptide provide a meaningful biological effect in vivo.

### Example 5—Reduction of Lesion Area by Aflibercept/Peptide Carrier Eye Drop Formulation in Mouse Laser Induced CNV Model

Laser photocoagulation in mouse eyes: Mice were anesthetized at 3.5% isoflurane with 1.0 L/min of oxygen and maintained at 2.5% isoflurane. Eyes were dilated with 3 uL tropicamide ophthalmic solution (0.5%; Akorn) for 1-2 min. The mice were placed on a heated stage and a drop of GenTeal Tears (Alcon) was added per eye prior to the start of laser photocoagulation. The center of the eye was lined up in the camera view and the camera was advanced toward the animal until the cornea touched the camera. The animal was adjusted until correctly aligned. Laser photocoagulation was performed using the Micron IV (Phoenix Technology Group) attached to an imaged-guided laser integrated with Discover software (Phoenix Technology Group). The laser power was calibrated to be between 18.2-18.7 mW prior to the beginning of each experiment when the laser was set at 50 mW, 2000 ms, and no light. The laser was used at 270 mW of power and 80 ms pulse duration. Burn spots were 50 um in diameter, placed at 12, 1:30, 6, and 9 o'clock, about two diameters away from the optic nerve, and away from blood vessels. After laser photocoagulation, animals recovered alone in a warm cage until ambulatory.

Choroidal Flatmount Preparation and Isolectin B4 Staining of CNV Lesions: After day-7 OCT and FFA image acquisition, animals were euthanized and enucleated. Eyes were washed in PBS and fixed in 4% paraformaldehyde for one hour at room temperature. After fixation, eyes were transferred to PBS. Under a dissection microscope, the anterior portion and lens of the eye were removed. The neural retina was carefully removed before making eight radial incisions on the posterior eye cup. The eye cups were blocked and permeabilized with PBS containing 0.2% Tween-20 and 0.5% BSA (Genesee Scientific, CA) for one hour on an orbital shaker. The blocking buffer was removed and replaced with IB4 stain buffer containing GS-IB₄ conjugated with Alexa Fluor 647 (5-10 ug/mL, Invitrogen, Thermo Fisher Scientific) in PBS with 1 mM calcium chloride. Samples were incubated overnight at 4° C. The following day, samples were washed multiple times with PBS and mounted with Fluoromount (Sigma-Aldrich, St. Louis, MO) on glass slides. CNV lesions were imaged using the Revolve microscope (ECHO, San Diego, CA). CNV lesion area was measured using ImageJ freehand selection tool (U.S. National Institute of Health, Bethesda, MD) with the scale set using a scale bar image from the Revolve microscope.

Optical Coherence Tomography (OCT) scan in mouse eyes: Mice were anesthetized at 3.5% isoflurane with 1.0 L/min of 02 and maintained at 2.5%. Eyes were dilated with 3 uL tropicamide ophthalmic solution (0.5%; Akorn) for 1-2 min. The mice were placed on a heated stage and a drop of Genteal Tears gel lubricant (Alcon) was added per eye prior to the start of optical coherence tomography (OCT) image acquisition. The center of the eye was lined up in the camera view and the camera was advanced toward the animal until the cornea touched the camera. The animal was adjusted until correct alignment. OCT line scan pattern images were acquired using the Micron IV attached to an imaged-guide OCT integrated with the Reveal software (Phoenix Technology Group). After imaging, animals recovered alone in a warm cage until ambulatory. Post-image acquisition analysis was performed using InSight (Phoenix Technology Group) software. A manual caliper was used to measure the following parameters of the lesion in micrometers: height, width, length, area (height*width), area (height*length), volume (height*width*length), and retinal thickness (line from nerve layer to the imaginary line of RPE). All statistics were done using GRAPHPAD PRISM 7 software (GraphPad Software Inc., San Diego, CA, USA). Two-way Repeated Measures ANOVA was done followed by Dunnett's multiple comparison post-hoc test where applicable with significance at P s 0.05. One-way ANOVA was done followed by Dunnett's multiple comparisons post hoc test where applicable with significance at P s 0.05.

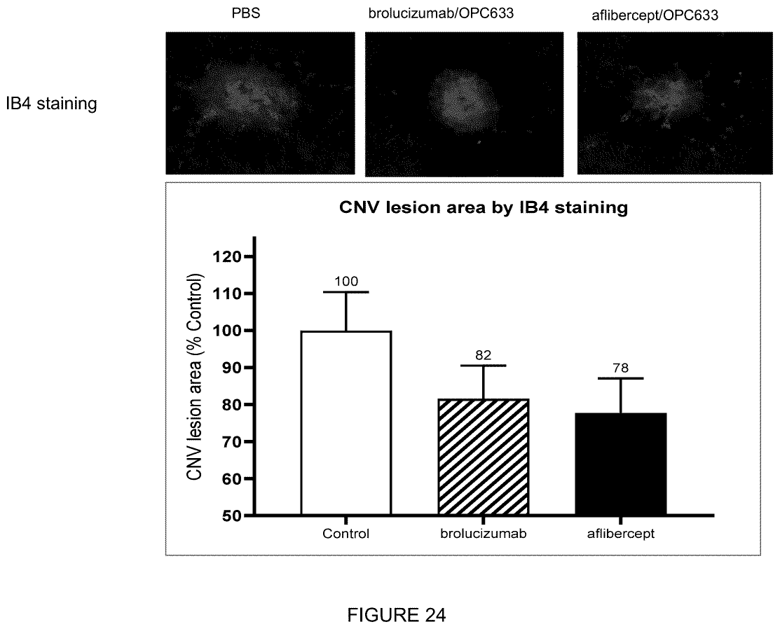

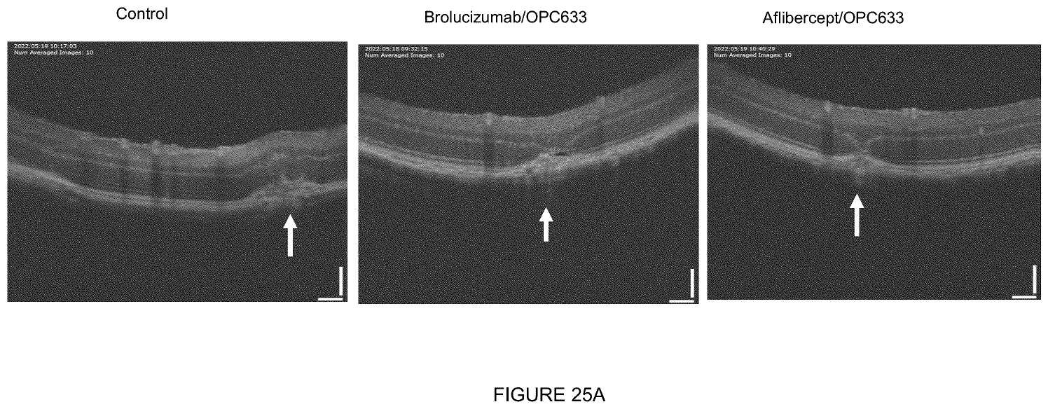

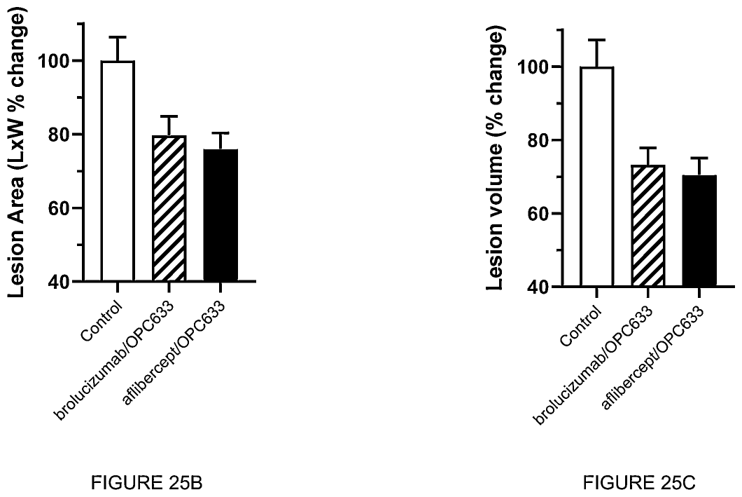

Results are shown in FIGS. 25A-25C. As shown in FIGS. 25B and 25C, a reduction in lesion area and lesion was observed in mice treated with the peptide carrier:brolucizumab and peptide carrier:aflibercept formulations compared to the control. See also FIG. 24. The data provided herein demonstrate the ability of the peptide carriers of the disclosure to deliver therapeutic antibodies into the intraocular space following topical administration such that a therapeutic effect is achieved in a clinically relevant animal model.

### Example 6—Reduction of Vascular Leakiness by Aflibercept/Peptide Carrier Eye Drop Formulation Vs IVT Injected Aflibercept in Mouse Laser Induced CNV Model

Fundus Fluoresceine Angiography (FFA) in mouse CNV lesions: Seven-day randomization: On the morning of the 8^{th} day post laser photocoagulation, mice were warmed in a warming cage until tail veins were dilated. Subsequently, mice were anesthetized at 3.5% isoflurane with 1.0 L/min of O₂ and maintained at 2.5% isoflurane. The tail was cleaned with an isopropyl alcohol pad. Using a 0.3 mL insulin syringe (BD), mice were intravenously injected with a 50 uL bolus of AK-Fluor at 1 mg/mL. Right and left eye images were acquired at 4 minutes and 6 minutes respectively post fluorescein injection on the Micron IV fundus camera set with a FITC-filter integrated with the Discover software (Phoenix Technology Group). Lesion images were assessed and graded on leakage area and signal intensity. Lesions with hemorrhages and no observed breaks of Bruch's membrane were automatically excluded. Lesions with the brightest signal and largest lesion areas were classified as Tier 1. Lesions with medium brightness were classified as Tier 2. All other lesions left were Tier 3. Only Tier 1 lesions were included in the study. The mice randomly redistributed so there were even numbers of Tier 1 lesions among all treatment groups.

Eye drops vs IVT injection: The formulated eye drops at 3 ul/eye were applied under anesthetic conditions (3.5% isoflurane at 1.5 L/min of oxygen), and the subjects were kept under anesthesia for 3 minutes before recovering alone in a warm cage until ambulatory. Five eye drops per day were applied between 8am-10 pm daily for 7 days. For intravitreal injections, pre-operatively the animal was given one dose of meloxicam at 1 mg/kg subcutaneously. The anesthetized animal was placed under a dissection microscope. A drop of topical anesthetic, proparacaine (Sandoz), was applied to each eye undergoing treatment. The eye was gently proptosed to reveal the equator of the globe. A 34G sharp needle affixed to a 1 ml syringe was inserted through the sclera approximately 1-2 mm below the limbus and withdrawn. A 36G sharp needle affixed to a nanofil syringe (WPI) carrying the test article was inserted through the pre-punctured hole at a 45° angle through the sclera into the vitreous body. The needle was carefully advanced between the retina and the lens, and the test article was slowly injected into the vitreous humor. The needle was withdrawn after holding a few seconds, and the eyes were treated with antibiotic ophthalmic ointment (Neosporin; Akorn). The animal then recovered alone in a warm cage until ambulatory.

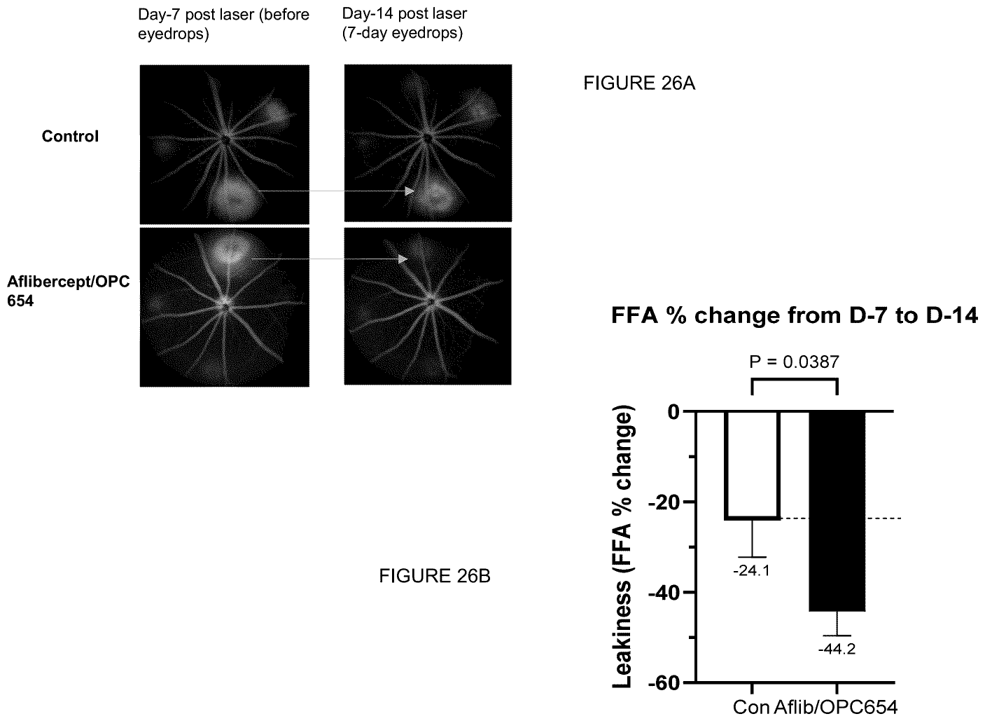

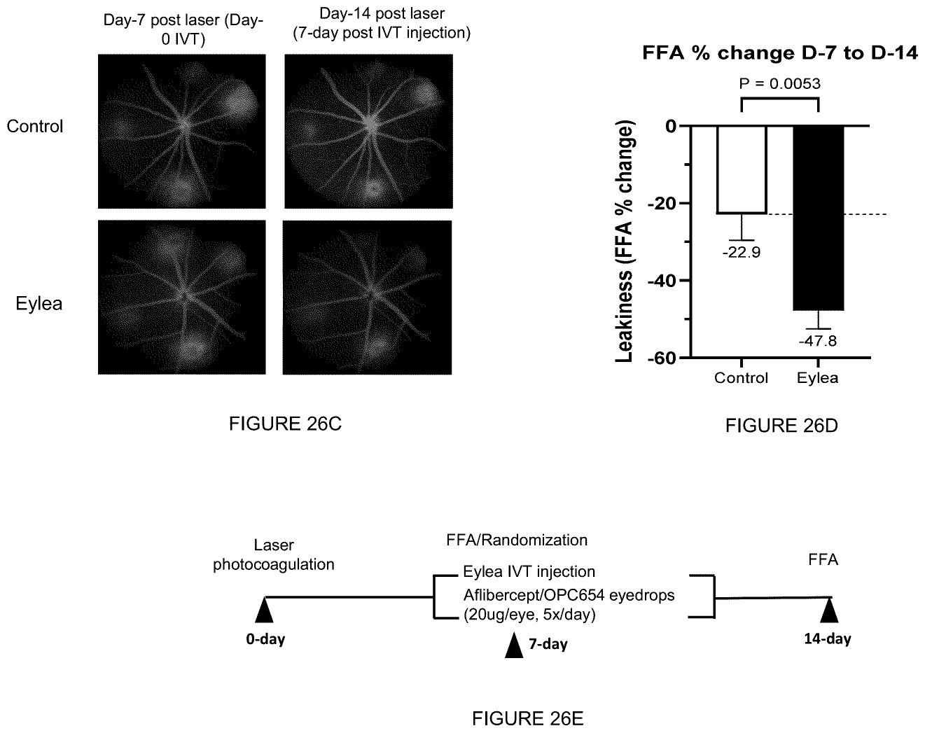

Results are illustrated in FIGS. 26A-26D. FIG. 26A shows a reduction in vascular leakiness by eye drops comprising aflibercept and peptide carrier 654; the cloud in the image is significantly reduced. The percent change in FFA was significantly less in the subject administered peptide carrier and payload peptide (see, e.g., FIG. 26B). The results achieved via topical administration of a peptide carrier/payload peptide of the disclosure were similar to that observed using intravitreal injection without a peptide carrier. See FIGS. 26C and 26D. The data reported in this example demonstrate that topical administration of the peptide carriers of the disclosure with payload peptide to the surface of the eye can achieve a therapeutic effect similar to that of intravitreal injection in a clinically relevant animal model.

### Example 7—Intraocular Penetration Activity of Various Constructs in Pig Eyes

Ocular penetration screen of antibody/carrier formulation in pig eyes: I R800-aflibercept biosimilar (PMC-902) was formulated with peptide carrier 631 in PBS. C57BL/6 (Charles River Laboratories, Wilmington, MA) and C57BL/6J (Jackson Laboratory Bar Harbor, ME) female mice were anesthetized with isoflurane. A 3 ul eye drop containing 5 ug IR800 labeled antibody was formulated with 20× molar concentration of peptide carrier and placed in an eye drop bottle. Each 50 ul eye drop contained 50 ug antibody with peptide carrier in PBS. Yorkshire pigs (12-16 weeks old) were placed in a sling before eye drop application. Two eye drops were placed per eye, followed by another dose of two eye drops one hour later. Animals were euthanized and enucleated two hours after the initial eye drops. Eyes were rinsed in PBS and fixed in 4% paraformaldehyde for 24 hours. Eyes were stored in PBS. Eyes were dissected and aqueous humor, vitreous, and retina were harvested into 24-well plates. Plates were scanned with an Odyssey CLx (LI-COR).

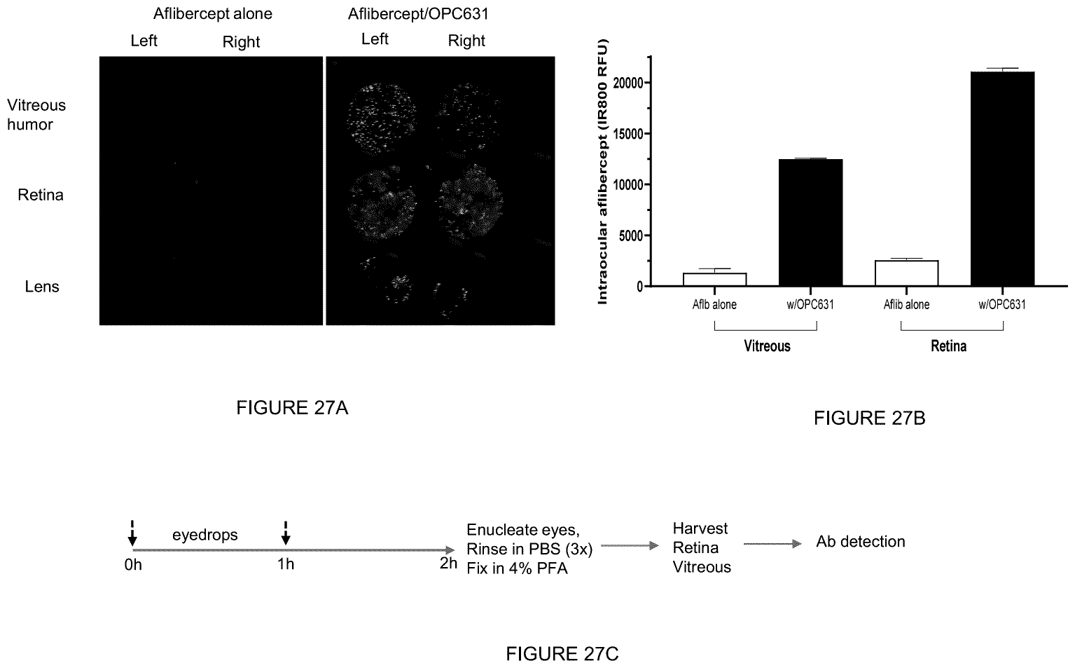

Results are shown in FIGS. 27A and 27B. Very little aflibercept was observed in vitreous humor, the retina, or the lens in the absence of peptide carrier. The peptide carrier dramatically enhanced penetration of the recombinant protein aflibercept biosimilar in vitreous humor and the retina of porcine eyes.

### Example 8—Detection of Dual Antibody Delivery into Mouse Eyes

To detect dual antibody delivery into mouse eyes via a single peptide carrier, eye drops were formulated in PBS containing 5 ug IR800 labeled PMC-902 and/or 3 ug IR680 labeled PMC-401s with one peptide carrier (here, peptide carrier 654 or 747). For dual antibodies, each antibody was formulated with the peptide carrier and incubated 10 minutes, then the two antibody compositions were combined. After eye drop application, animals were euthanized, enucleated, and the harvested vitreous and retina tissues were homogenized in RIPA buffer supplemented with protease inhibitor cocktail (Sigma-Aldrich). Clarified lysates were mixed with loading buffer (LI-COR) and loaded onto a 4-20% polyacrylamide gel for SDS-PAGE (BioRad). Gels were scanned using the Odyssey CLx (LI-COR).

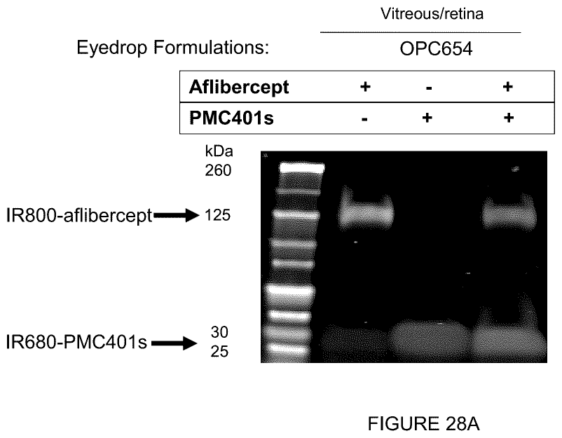

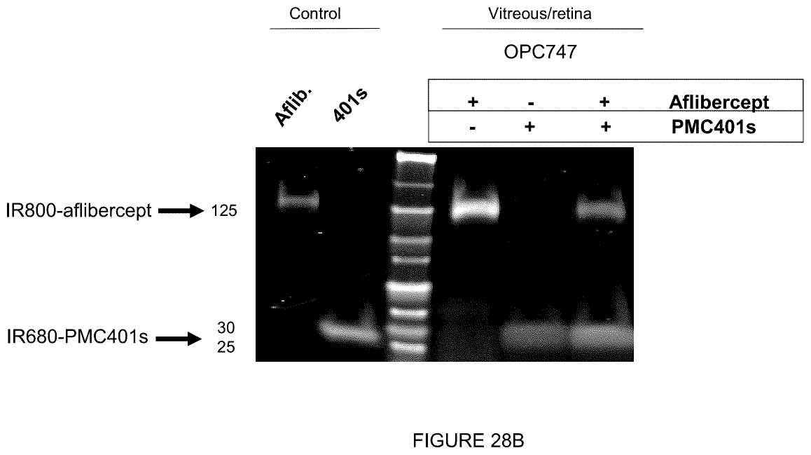

Results are shown in FIGS. 28A and 28B. Both tested peptide carriers mediated intraocular penetration of both antibodies, separately and when the antibodies were administered together in the same composition.

All patents and other publications cited herein are expressly incorporated herein by reference in their entireties.

Sequence list:

| SEQ | |
|---|---|
| ID | |
| NO: | SEQUENCE |
| 1 | LKKRKVVRLI KFLLKFFLIPKG LVGVFH |
| 2 | LKKRKVVRLI KFLLKMFFLIPKG LVGVFH |
| 3 | IKKRKVVRLI KFLLKLFFLIPKG LVGVFH |
| 4 | VKKRKVVRLI KFLLKLFFLIPKG LVGVFH |
| 5 | MKKRKVVRLI KELLKLFFLIPKG LVGVFH |
| 6 | MKKRKVVRLI KYLLKLFFLIPKG LVGVFH |
| 7 | IKRRKVVRLI KFLLKLFFLIPKG LVGVFH |
| 8 | IKKRKVVRLI KYLLKLFFLIPKG LVGVFH |
| 9 | IKKRKVVRLI KKLLKLFFLIPKG LVGVFH |
| 10 | IKKRKVLRLI KYLLKLFFLIPKG LVGVFH |
| 11 | IKKRKVLRLI KYLVKLFFLIPKG LVGVFH |
| 12 | IKKRKIIRLI KFLLKLFFLIPKG LVGVFH |
| 13 | IKKRKIVRLI KFLVKLFFLIPKG LVGVFH |
| 14 | LKKRKVVRLI KFLLFFLIPKG LVGVFH |
| 15 | LKKRKVVRLI KFLFFLIPKG LVGVFH |
| 16 | LKKRKVVRLI KFFFLIPKG LVGVFH |
| 17 | LKKRKVVRLI KFLLKLLVGVFHFFLIPKG |
| 18 | FFLIPKG LKKRKVVRLI KFLLKLLVGVFH |
| 19 | VKKRKIVRLI KFLVKLFFLIPKG LVGVFH |
| 20 | VKKRKIVRLI KFLVKMFFLIPKG LVGVFH |
| 21 | VKKRKIVRLI KFLLKFFLIPKG LVGVFH |
| 22 | VKKRKVVRLI KFLLKFFLIPKG LVGVFH |
| 23 | VKKRKVVRLI KLFFLIPKG LVGVFH |
| 24 | VKKRKVVRLI KYFFLIPKG LVGVFH |
| 25 | VKKRKIVRLI KVFFLIPKG LVGVFH |
| 26 | VKKRKVVRLI KFVLKFFLIPKG LVGVFH |
| 27 | VKKRKVVRLI KFVLKYFLIPKG LVGVFH |
| 28 | VKKRKLVRLI KFVLKFFLIPKG LVGVFH |
| 29 | VKKRKIVRLI KFVLKFFLIPKG LVGVFH |
| 30 | VKKRKIVRLI KFFLIPKG LVGVFH |
| 31 | VKKRKIVRLI FFLIPKG LVGVFH |
| 32 | VKKRKIVRLI KFLLKFFLIPKG IVGVFH |
| 33 | VKKRKIVRLI KFLLKYFLIPKG VVGVFH |
| 34 | VKKRKVVRLI KYFFLIPKG VVGVFH |
| 35 | VKKRKIVRLI KYFFLIPKG VVGVFH |
| 36 | VKKRKIVRLI KYFFLVPKG IVGVFH |
| 37 | VKKRKVVRLI KYYFLIPKG IVGVFH |
| 38 | VKKRKVIRLI KFFLIPKG LVGVFH |
| 39 | VKKRKILRLI KFFLIPKG LVGVFH |
| 40 | VKKRKIVRLI KFFLIPK LVGVFH |
| 41 | LKKRKVVRLI KFLLKLLKKR KVVFFLIPKG RRRRRR |
| 42 | LKKRKVVRLI KFLLKLLKKR KVVFFLIPKG RRRR |
| 43 | LKKRKVVRLI KELLKL FFLIPKG RRRR |
| 44 | LKKRKVVRLI KFLLKLLKK FFLIPKG RRRR |
| 45 | LKKRKVVRLI KFLLKLLKKFFLIPKG ACSSSPSKHCG |
| 46 | LKKRKVVRLI KELLKL FFLIPKG RPARPAR |
| 47 | LKKRKVVRLI KFLLKLFFLIPKG THRPPMWSPVWP |
| 48 | LKKRKVVRLI KFLLKLFFLIPKG GYRPVHNIRGHWAPG |
| 49 | LKKRKVVRLI KFLLKFFLIPKG RLYE |
| 50 | LKKRKVVRLI KFLLKFFLIPKG RPARPAR |
| 51 | LKKRKVVRLI KFLLKLFFLIPKG AHLHNRS |
| 52 | LKKRKVVRLI KFLLKLFFLIPKG NAHQARST |
| 53 | LKKRKVVRLI KFLLKLFFLIPKG LVGVFH |
| 54 | LKKRKVVRLI KFLLKLFFLIPKG QHREDGS |
| 55 | LKKRKVVRLI KFLLKLFFLIPKG RPLKPW |
| 56 | LKKRKVVRLI KFLLKLLVFFLIPKG RLYE |
| 57 | LKKRKVVRLI KFLLKLFFLIPKG NLLMAAS |
| 58 | LKKRKVVRLI KFLLKLFFLIPKG DKPRR |
| 59 | LKKRKVVRLI KFLLKLFFLIPKG RPAR |
| 60 | LVGVFH LKKRKVVRLI KFLLKLFFLIPKG |
| 61 | LVGVFH LKKRKVVRLI KFLLKFFLIPKG |
| 62 | LVGVFH LKKRKVVRLI KFLLFFLIPKG |
| 63 | LVGVFH LKKRKVVRLI KFLFFLIPKG |
| 64 | LVGVFH LKKRKVVRLI KFFFLIPKG |
| 65 | LVGVFH VKKRKIVRLI KFLVKFFLIPKG |
| 66 | IVGVFH VKKRKIVRLI KFLVKFFLIPKG |
| 67 | LVGVFH VKKRKIVRLI KFFFLIPKG |
| 68 | IVGVFH VKKRKIVRLI KFYFLIPKG |
| 69 | IVGVFH VKKRKIVRLV KFYFLIPKG |
| 70 | LVGVFH VKKRKVVRVI KFFFLIPKG |
| 71 | VVGVFH VKKRKIVRLI KFLLKFFLIPKG |
| 72 | VVGVFH VKKRKIVRLI KFFFLIPKG |
| 73 | RRRRRR |
| 74 | RKKRRQRRR |
| 75 | KKLFKKILKYLKKLFKKILKYL FFLIPKG RRRRRR |
| 76 | LKKRKVVRLI KFLLKLLKKR KVVFFLIPKG |
| | ACSSSPSKHCG |
| 77 | KKLFKKILKY LINLKALAAL AKKIL RPSRPRIRYKC |
| 78 | LKKRKVVRLI KFLLKLLKKR KVVFFLIPKG RRRRRR |
| | AVPF |
| 79 | LKKRKVVRLI KFLLKLLKKR KVVFFLIPKG MASIWVGHRG |
| | AVPF |
| 80 | LKKRKVVRLI KFLLKLLKKR KVVFFLIPKG RRRR RLYE |
| 81 | LKKRKVVRLI KFLLKLLKKR KVVFFLIPKG RLYE |
| 82 | KKLFKKILKY LKKLFKKILK YLRPSRPRIRYKC RPARPAR |
| 83 | LKKRKVVRLI KFLLKLLKKR KVVFFLIPKGRRRRPARPAR |
| 84 | LKKRKVVRLI KFLLKLLKKR KVVFFLIPKG RRRR |
| | RRKRRR |
| 85 | LKKRKVVRLI KFLLKLLKKR KFFLIPKG ACSSSPSKHCG |
| | RRKRRR |
| 86 | LKKRKVVRLI KFLLKLLKKR KFFLIPKG ACSSSPSKHCG |
| 87 | LKKRKVVRLI KNLLKLLKKR KVNFFLIPKG RRRR |
| 88 | LKKRKVVRLI KFNLKLLKKR KVNFFLIPKG RRRR |
| 89 | LKKRKVVRLI KFLLKLFFLIPKG ACSSSPSKHCG |
| 90 | KKRKVVRLI KFLLKLLKKR KVVFFLIPKG |
| 91 | LKKRKVVRLI KFLLKLLKKR KVVFFLIPKG E |
| 92 | LKKRKVVRLI KFLLKLLKKR KVVFFLIPKG LE |
| 93 | LKKRKVVRLI KFLLKLFFLIPKG RLYE |
| 94 | LKKRKVVRLI KFLLKLFFLIPKG HHIYLGAVNYIY |
| 95 | LKKRKVVRLI KFLLKLLKKFFLIPKG LE |
| 96 | LKKRKVVRLI KFLLKLRFFLIPKG RLYE |
| 97 | KAFDITYVRLKFFLIPKG |
| 98 | FFLIPKG LVGVFH LKKRKVVRLI KFLLKL |
| 99 | XXXXXXXXXXXXXXXXXF-L-I/V-P-K-G |
| 100 | XXXXXXXXXXXXXXXXXF-L-I-P-K-G |
| 101 | L/V-K-K-R-K-I/V-V-R-L-I-K-XXXXXFFLIPKG |
| 102 | LVGVFH |
| 103 | RRRRRR |
| 104 | RRRR |
| 105 | RYLE |
| 106 | RPARPAR |
| 107 | MASIWVGHRGAVPF |
| 108 | RPSRPRIRYKC |
| 109 | RRKRRR |
| 110 | ARPCA |
| 111 | GVITRIR |
| 112 | ACSSSPSKHCG |
| 113 | LVGVFH |
| 114 | HAIYPRH |
| 115 | GYRPVHNIRGHWAPG |
| 116 | AHLHNRS |
| 117 | NAHQARST |
| 118 | QHREDGS |
| 119 | NLLMAAS |
| 120 | DKPRR |
| 121 | RPAPR |
| 122 | CNGRCG |
| 123 | THRPPMWSPVWP |
| 124 | RPLKPW |
| 125 | KAFDITYVRL KFFLIPKGRP ARPAR |
| 126 | VKKRKIVRLI KFFLIPKLVG VFHRLYE |
| 127 | KKLKKLKVRL VKFLLKFGPL RYRVVLFIER IKLK |
| 128 | KKLKKLKVRL VKFLLKFGPL RYRVVL |

What is claimed is:

1. A peptide comprising no more than 50 amino acids and comprising the amino acid sequence of Formula (1):
X1-X2-X3-X4-X5-X6-X7-X8-X9-X10-X11-X12-X13-X14-X15-X16-X17-F-L-I/V-P-K-G, wherein
X1 is V, M, L, I, or K;
X2 is K, R, or H;
X3 is K, R, or H;
X4 is R, K, or H;
X5 is K, R, or H;
X6 is I, V, or L;
X7 is V, L, or I;
X8 is R, K, or H;
X9 is L, K, V, or R;
X10 is I, L, or V;
X11 is K, R, or H;
X12 is F, Y, K, or L, or is absent;
X13 is L, V, or I, or is absent;
X14 is V, L, or I, or is absent;
X15 is K, L, R, or H, or is absent;
X16 is (i) M, L, V, I, R, A, K, G, C, Y, S, N, or D; is an amino acid of (i) in combination with L, K, R, or V, or (iii) is absent; and
X17 is F, Y, FL, YL, FLK, YLK, FLKL, or YLKL.

2. The peptide of claim 1, wherein the peptide comprises the amino acid sequence of Formula (2): X1-X2-X3-X4-X5-X6-X7-X8-X9-X10-X11-X12-X13-X14-X15-X16-X17-F-L-I-P-K-G.

3. The peptide of claim 2, wherein the peptide comprises the amino acid sequence of Formula (3): L/V-K-K-R-K-I/V-V-R-L-I-K-X12-X13-X14-X15-X16-F-F-L-I-P-K-G, wherein
X12 is F or Y or is absent;
X13 is L or is absent;
X14 is L or V or is absent;
X15 is K or is absent; and
X16 is L or M or is absent.

4. The peptide of claim 1, further comprising, attached at the N-terminus, L, L-L, or the amino acid sequence of Formula (4): X30-V/L-G-V/L-F-H, wherein X30 is L, I, or V.

5. The peptide of claim 4, wherein the amino acid sequence attached to the N-terminus is L-V-G-V-F-H (SEQ ID NO: 102).

6. The peptide of claim 1, further comprising the amino acid sequence of one of SEQ ID NOs: 103-124, attached at the C-terminus.

7. The peptide of claim 1, wherein the peptide comprises the amino acid sequence set forth in any one of SEQ ID NOs: 1-72 comprising one, two, or three amino acid substitutions within the amino acid sequence.

8. The peptide of claim 1, wherein the peptide comprises the amino acid sequence set forth in any one of SEQ ID NOs: 1-72.

9. The peptide of claim 1, wherein the peptide comprises the amino acid sequence set forth in any one of SEQ ID NOs: 20, 21, 24, 30, 53, 60, 67, and 72.

10. The peptide of claim 1, wherein the peptide comprises the amino acid sequence set forth in any one of SEQ ID NOs: 1-40.

11. The peptide of claim 1, wherein the peptide comprises an amino acid sequence set forth in any one of SEQ ID NOs: 41-59.

12. The peptide of claim 1, wherein the peptide comprises the amino acid sequence set forth in any one of SEQ ID NOs: 60-72.

13. A construct comprising the peptide carrier of claim 1 conjugated to one or more payload peptides.

14. The construct of claim 13, wherein the payload peptide is an antibody or comprises an antigen-binding antibody fragment.

15. The construct of claim 13, wherein the payload peptide is a recombinant protein.

16. The construct of claim 13, wherein the payload peptide is aflibercept, ranibizumab, brolucizumab, bevacizumab, faricimab, ramucirumab, fresolimumab, cetuximab, panitumumab, asunercept, recCD59, NGM621 or ANX007.

17. A composition comprising the construct of claim 13 and a pharmaceutically acceptable carrier, diluent or excipient.

18. A composition comprising a peptide carrier of claim 1, a payload peptide, and a pharmaceutically acceptable carrier, diluent or excipient.

19. The composition of claim 17, which is formulated as an eye drop.

20. A method of delivering a payload peptide to an eye of a subject, the method comprising administering the composition of claim 17 to the eye of the subject.

21. A method of treating an ocular disorder in a subject in need thereof, the method comprising administering the composition of claim 17 to an eye of the subject.

22. The method of claim 21, wherein the ocular disorder is wet age-related macular degeneration, diabetic macular edema, diabetic retinopathy, retinal vein occlusion, geographic atrophy, uveitis, endophthalmitis, retinal detachment, optic neuropathy, optic neuritis, neuromyelitis optica, retinitis pigmentosa or uveal melanoma.

23. The method of claim 20, wherein the composition is topically administered to the subject.

24. A peptide comprising the amino acid sequence set forth in any one of SEQ ID NOs: 75-98 wherein one, two, or three amino acids within the amino acid sequence are substituted.

25. The peptide of claim 24, comprising the amino acid sequence of any one of SEQ ID NOs: 75-98.

26. A peptide comprising the amino acid sequence set forth in any one of SEQ ID NOs: 125-128 wherein one, two, or three amino acids within the amino acid sequence are substituted.

27. The peptide of claim 26, comprising the amino acid sequence of any one of SEQ ID NOs: 75-98.
